(19) United States
(12) Patent Application Publication
(10) Pub. No.: US 2016/0095867 A1
(43) Pub. Date: Apr. 7, 2016

(54) BET INHIBITION THERAPY FOR HEART DISEASE

(71) Applicants: DANA-FARBER CANCER INSTITUTE, INC., Boston, MA (US); CASE WESTERN RESERVE UNIVERSITY, Cleveland, OH (US); THE BRIGHAM AND WOMEN'S HOSPITAL, INC., Boston, MA (US)

(72) Inventors: Saptarsi M. Haldar, Pepper Pike, OH (US); James E. Bradner, Weston, MA (US); Jonathan D. Brown, S. Hamilton, MA (US)

(73) Assignees: DANA-FARBER CANCER INSTITUTE, INC., Boston, MA (US); CASE WESTERN RESERVE UNIVERSITY, Cleveland, OH (US); THE BRIGHAM AND WOMEN'S HOSPITAL, INC., Boston, MA (US)

(21) Appl. No.: 14/894,040

(22) PCT Filed: May 28, 2014
(86) PCT No.: PCT/US14/39790

§ 371 Date: Nov. 25, 2015

Related U.S. Application Data
(60) Provisional application No. 61/828,166, filed on May 28, 2013.
(60) Provisional application No. 61/931,062, filed on Jan. 24, 2014.

(51) Int. Cl. A61K 31/5517; A61L 31/08; A61L 31/16; A61K 9/00

(52) U.S. Cl. CPC A61K 31/5517; A61K 9/0034; A61L 31/08; A61L 31/16

(57) ABSTRACT

Method for treating cardiac diseases using BET inhibitors including JQ1 are provided. Methods for treating cardiac hypertrophy, method for treating heart failure not arising from inflammation, methods for treating myocardial infarction, methods for cardioprotection and methods for inhibiting restenosis are described herein. The methods involve the use of effective amounts of BET inhibitors such as JQ1.

### RELATED APPLICATIONS

This application claims the benefit under 35 U.S.C. §119(e) of U.S. provisional application Ser. No. 61/828,166, filed May 28, 2013, and U.S. provisional application Ser. No. 61/931,062, filed Jan. 24, 2014, the contents of which are incorporated by reference herein in their entirety.

### FEDERALLY SPONSORED RESEARCH

This invention was made with Government support under National Institute of Health Grant R01 DK093821. Accordingly, the Government has certain rights in this invention.

### BACKGROUND OF THE INVENTION

Heart failure (HF) is a leading cause of mortality, hospitalization, and healthcare expenditures in modern society. This disease occurs when the heart is unable to maintain organ perfusion at a level sufficient to meet tissue demand, and results in fatigue, breathlessness, multi-organ dysfunction, and early death. Existing pharmacotherapies for individuals afflicted with HF, such as beta adrenergic receptor antagonists and inhibitors of the renin-angiotensin system, generally target neurohormonal signaling pathways. While such therapies have improved survival in HF patients, residual morbidity and mortality remain unacceptably high. In light of this major unmet clinical need, the elucidation of novel signaling pathways involved in HF pathogenesis holds the promise of identifying new therapies for this highly prevalent and deadly disease.

### SUMMARY OF THE INVENTION

The invention, relates in some aspects to the discovery that BETs (bromodomain and extraterminal family of bromodomain-containing reader proteins) are critical effectors of pathologic cardiac remodeling via their ability to co-activate a broad, but defined stress-induced transcriptional program in the heart. In addition, the inventors of the present application have discovered that BET inhibitors, such as JQ1, can surprisingly, inhibit muscle cell growth in connection with cardiac hypertrophy and blood vessel injury. Accordingly, some aspects of the invention involve a method of treating cardiomyopathy by administering to a subject in need to such treatment an amount of a compound of the invention, e.g., JQ1 effective to treat the cardiomyopathy.

In some embodiments, the subject does not have heart failure. In some embodiments, the subject is free of symptoms of obstructive coronary artery disease. In some embodiments, the subject is not being treated for atherosclerosis. In some embodiments, the subject is not being treated for obstructive coronary artery disease, as evidenced by an angiogram showing. In some embodiments, the subject does not have heart failure or atherosclerosis and is not recovering from a myocardial infarction. In some embodiments, the subject is receiving therapy for reducing blood pressure. In some embodiments, the cardiomyopathy is due to chronic hypertension, valvular heart disease (includes aortic valve stenosis, aortic valve insufficiency, mitral valve insufficiency), peripartum cardiomyopathy, or cardiomyopathy due to genetic mutations (includes familial hypertrophic cardiomyopathy and familial dilated cardiomyopathy). In some embodiments, the compound of the invention is JQ1. In some embodiments, the cardiomyopathy is cardiac hypertrophy.

According to one aspect of the invention, a method for treating heart failure not arising from inflammation is provided. The method comprises administering to a subject in need of such treatment an amount of a compound of the invention, e.g., JQ1, effective to treat the heart failure. In some embodiments, the subject does not have obstructive coronary artery disease, as evidenced by an angiogram showing. In some embodiments, the subject is not recovering from a myocardial infarction. In some embodiments, the heart failure is due to:

(i) Heart failure with preserved ejection fraction (HFpEF) without evidence of obstructive coronary artery disease;

(ii) Heat failure due to toxicity of drugs (including anti-cancer agents and drugs of abuse);

(iii) Heart failure caused by ethanol abuse;

(iv) Heart failure due to chronic tachycardia (rapid heart rate);

(v) Heart failure due to endocrine abnormalities (excessive thyroid hormone, growth hormone, diabetes, pheochromocytoma);

(vi) High-output heart failure (includes that which is caused by anemia or peripheral atriovenous shunting);

(vii) Heart failure caused by nutritional deficiencies (including thiamine, selenium, calcium, and magnesium deficiency);

(viii) Heart failure due to viral infection (including HIV) or

(ix) Heart failure due to congenital heart malformations.

In some embodiments, the subject is receiving therapy for reducing blood pressure. In some embodiments, the compound of the invention is JQ1.

According to some aspects of the invention, a method for treating myocardial infarction is provided. The method involves administering to a subject in need of such treatment a compound of the invention, e.g., JQ1, in an amount effective to treat the myocardial infarction, wherein the compound of the invention, e.g., JQ1, administration is initiated not sooner than 5 days after the myocardial infarction. In some embodiments, the compound of the invention, e.g., JQ1, administration is initiated not sooner than 6 days after the myocardial infarction. In some embodiments, the compound of the invention, e.g., JQ1, administration is initiated not sooner than 7 days after the myocardial infarction. In some embodiments, the subject does not have atherosclerosis as evidenced by an angiogram showing. In some embodiments, the subject does not have heart failure. In some embodiments, the compound of the invention is JQ1.

According to some aspects of the invention, a method for cardioprotection is provided. The method comprises administering to a subject receiving a therapy that is cardio toxic a BET inhibitor in an amount effective to inhibit cardio toxicity by such therapy. In some embodiments, the therapy is anti-cancer therapy. In some embodiments, the anti-cancer therapy is chemotherapeutic therapy. In some embodiments, the chemotherapeutic is an anti-cancer agent selected from the group consisting of anthracyclines, trastuzumab, 5-fluorouracil, mitoxantrone, paclitaxel, vinca alkaloids, tamoxifen, cyclophosphamide, imatinib, trastuzumab, capecitabine, cytarabine, sorafenib, sunitinib, and bevacizumab. In some embodiments, the BET inhibitor is a JQ1 molecule.

According to some aspects of the invention, a method for inhibiting restenosis is provided. The method comprises administering to a subject undergoing an angioplasty and/or receiving a stent a BET inhibitor in an amount effective to inhibit restenosis. In some embodiments, the BET inhibitor is administered locally at the site of a stenosis. In some embodiments, the BET inhibitor is administered via a catheter. In some embodiments, the BET inhibitor is administered as an element of a coating on a stent. In some embodiments, the BET inhibitor is a JQ1 molecule.

Some aspects of the invention provide a stent for preventing stenosis or restenosis, the stent including a coating for delivering a drug agent locally to the vasculature when the stent is positioned at the vasculature, wherein the improvement comprises a BET inhibitor included in the coating. In some embodiments, the BET inhibitor is a JQ1 molecule.

In any of the foregoing embodiments, the compounds of the invention are compounds of Formulae I-XXII described herein and in WO 2011/143669 which is incorporated by reference herein. In some important embodiments, the compounds of the invention are compounds of Formulae I-IV. In preferred embodiments, the compound of the invention is JQ1.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 shows BET expression in the heart. (A) Relative expression of indicated BET genes by qRT-PCR in (A) NRVM and (B) adult mouse heart tissue (N=4). P<0.05 vs. Brd2 and Brd3. (C) Western blot demonstrating presence of BRD4 in NRVM whole cell extracts (left) and in adult mouse and human heart tissue nuclear protein extracts (right). Tubulin and POL2 shown for loading. (D) Immunofluorescence staining in NRVM for BRD4, α-actinin, DAPI. Merged image demonstrates nuclear localized BRD4 signal. White bar=10 μM. (E) Western blot demonstrating effective knockdown of BRD4 protein in NRVM with densitometric quantification (N=3). *P<0.05 vs. sh-cntrl. (F) Chemical structures of BET inhibitors used in FIG. 2F.

FIG. 2 shows that BET bromodomain inhibition blocks cardiomyocyte hypertrophy in vitro. (A) Chemical structure of (+)-JQ1. (B) Representative image of NRVM treated with or without JQ1 (250 nM) and PE (100 μM) for 48 hours with quantification of cardiomyocyte area. α-actinin immunofluorescence staining in green, DAPI in blue. *P<0.05 vs. DMSO −PE. **P<0.05 vs. JQ1 −PE. #P<0.05 vs. DMSO +PE. (C) qRT-PCR of hypertrophic marker genes in NRVM treated with JQ1 (500 nM) and PE (100 μM) for 48 h (N=4). #P<0.05 vs. veh, *P<0.05 vs. PE. (D) Representative image of NRVM infected with adenovirus containing sh-Brd4 or sh-cntrl (scrambled shRNA) treated with or without PE (100 μM) for 48 hours with quantification of cardiomyocyte area. *P<0.05 vs. sh-cntrl −PE. **P<0.05 vs. sh-Brd4 −PE. #P<0.05 vs. sh-cntrl +PE. (E) qRT-PCR of hypertrophic marker genes in NRVM treated with JQ1 (500 nM) and PE (100 μM) for 48 h (N=4). #P<0.05 vs. sh-cntrl, *P<0.05 vs. sh-cntrl+PE. (F) Cell area measurements of NRVM treated with a panel of structurally distinct BET inhibitors (JQ1, iBET, iBET-151, RVX-208, PF-1; 500 nM) and PE (100 μM) for 48 hours. *P<0.05 vs. −PE control for indicated compound. #P<0.05 vs. veh +PE. White bar=30 μM. NRVM area quantification performed from N=150-200 cardiomyocytes pooled from 3 independent experiments per group.

FIG. 3 shows that gene expression profiling defines BET regulated transcriptional programs during cardiomyocyte hypertrophy in vitro. (A) Selected heat map of differentially expressed transcripts. NRVM treated with 500 nM JQ1 and 100 μM PE. (B) Global analysis of differentially expressed transcripts showing induction of genes by PE with time and progressive reversal of PE-mediated gene induction by JQ1. (C) Volcano plot showing individual PE induced transcripts with suppression the same transcripts by JQ1. Location of 116 is annotated. (D) Functional pathway analysis (DAVID) of the panel of genes that were induced with PE and reversed by JQ1. False discovery rate (FDR) of <5% was considered statistically significant. (E) qRT-PCR of 116 from NRVM treated with JQ1 (500 nM) and PE (100 μM) for indicated timepoints (N=4). *P<0.05 vs. veh, #P<0.05 vs. PE. (F) ChIP-qPCR against Pol II and BRD4 in NRVM treated with JQ1 (500 nM) and PE (100 μM) for 90 min. PCR performed in vicinity of 116 transcriptional start site (TSS). −4 kb locus serves as nontarget region (location of PCR primers depicted above, N=3). *P<0.05 vs. veh, #P<0.05 vs. PE. (G) qRT-PCR of c-myc from NRVM treated with JQ1 (500 nM) and PE (100 μM) for indicated timepoints (N=4). While JQ1 suppresses 116 induction, it does not suppress c-myc induction. *P<0.05 vs. veh, #P<0.05 vs. PE.

FIG. 4 shows that BET expression in NRVM is invariant with PE stimulation. (A) Relative expression of Brd2-4 genes by qRT-PCR in NRVM treated with PE (100 μM) for indicated timepoints (N=4).

FIG. 5 demonstrates that BET Bromodomain inhibition with JQ1 potently attenuates pathologic cardiac hypertrophy and heart failure in vivo. (A) Experimental protocol for TAC and JQ1 administration in mice. (B) Echocardiographic parameters in mice during TAC (N=7; sham groups shown in FIG. 6). LVIDd is left ventricular end diastolic area, (IVS+PW)d is the sum thickness of the interventricular septum and posterior LV wall at end diastole. *P<0.05 vs. veh TAC. (C) Representative M-mode tracings and (D) end-diastolic 2D images of mice subject to 4 weeks TAC. White bar=2 mm. (E) Heart weight/body weight (HW/BW) and (F) Lung weight/body weight (LW/BW) ratios in mice after 4 weeks TAC (N=7 TAC, N=5 sham). *P<0.05 vs. sham veh. #P<0.05 vs. TAC veh. **P<0.05 vs. sham JQ1. (G) Representative photographs of freshly excised whole hearts from mice. Black bar=3 mm. (H) qRT-PCR of indicated genes from hearts of mice (N=5-7). *P<0.05 vs. sham veh. #P<0.05 vs. TAC veh. (I) JQ1 blocks PE-induced cardiac hypertrophy in vivo without compromising LV systolic function. Experimental protocol for PE infusion (75 mg/kg/day via subcutaneous osmotic minipump) and JQ1 administration in mice (N=7 PE, N=5 normal saline). See also FIG. 6.

FIG. 6 shows that JQ1 is well tolerated in mice and does not affect blood pressure or trans-aortic gradient. (A) Mice were given JQ1 (50 mg/kg/day IP) vs. vehicle for 17 days. Mice were subject to treadmill exercise (15 m/min, no incline) and time to exhaustion was measured (N=6). NS denotes statistical non-significance. (B) Echocardiographic parameters in sham treated mice (N=5). (C) Systolic blood pressure in mice treated with JQ1 (50 mg/kg/day IP) vs. vehicle for 17 days (N=5). (D) Pressure gradient across the surgically constricted segment of the aortic arch in mice 7 days after TAC (N=4).

FIG. 7 shows that BET Bromodomain inhibition in vivo blocks the development of cardinal histopathological features of heart failure. (A) Wheat germ agglutinin staining of heart sections and cardiomyocyte area quantification. Bar=30 μm. (B) Masson's Trichrome staining of heart sections with quantification of fibrotic area. Bar=400 μm for low magnification images (top) and 40 μm for high magnification images (bottom). (C) TUNEL staining of heart sections with quantification of TUNEL-positive nuclei below. Bar=20 μm. (D) PECAM-1 immunofluorescence staining of heart sections with quantification of myocardial capillary density. Bar=30 μm. For panels A-D: N=3-4, issues obtained from mice at 4 week timepoint, *P<0.05 vs. sham veh, #P<0.05 vs. TAC veh.

FIG. 8 shows that BETs co-activate a broad, but specific transcriptional program in the heart during TAC. (A) Protocol for microarray GEP experiment. (B) Unsupervised hierarchical clustering of gene expression profiles. (C) Heatmap of selected genes. (D) GEDI plots showing temporal evolution of gene clusters. (E) Volcano plot of individual transcripts. Genes that are induced with TAC are suppressed by JQ1. (F) Functional pathway analysis (DAVID) of the panel of genes that were induced with TAC and reversed by JQ1. A False discovery rate (FDR) of <5% was considered statistically significant. (G) GSEA for TAC-veh and TAC-JQ1 against three independent GEPs driven by cardiomyocyte-specific activation of nodal pro-hypertrophic transcriptional effectors in vivo: Calcineurin-NFAT (driven by a constitutively active Calcineurin A transgene (Bousette et al., 2010)), NFκB driven by an IKK2 transgene (Maier et al., 2012) and transgenic GATA4 overexpression (Heineke et al., 2007). FWER P<0.250 was considered to represent statistically significant enrichment. Data representative for all 3 timepoints and representative plots shown for 28 day timepoint. See also FIG. 9.

FIG. 9 shows the gene expression profiles of mouse hearts during TAC. (A) Relative expression of Brd2-4 by qRT-PCR in mouse hearts after sham/TAC at indicated timepoints (N=5-7). (B) Volcano plot (C) GSEA showing upregulation of c-myc targets with TAC-veh but no overlap with JQ1 effect. (D) qRT-PCR from hearts of mice at indicated timepoints (N=5-7) shows that JQ1 does not suppress c-myc induction. *P<0.05 vs. sham veh. #P<0.05 vs. sham JQ1.

FIG. 10 shows that BET regulated genes in the TAC model are relevant to human heart failure. (A) Venn diagram showing intersection of TAC-inducible genes that were suppressed by JQ1 against expression profile of genes upregulated in advanced non-ischemic and ischemic heart failure in humans (Hannenhalli et al., 2006). Targets of BETs in the mouse TAC model overlapped in a statistically significant manner with the set of genes induced in human heart failure (χ²<2×10⁻¹⁴). (B) Gene names populating the intersection of all 3 sets are listed.

FIG. 11A shows the study design. Adult mice were subject to pressure overload using transverse aortic constriction (TAC). JQ1 or vehicle was begun on day 18 post-TAC, a time point when significant pathology has already developed. JQ1 significantly attenuates the progression of (B) LV systolic dysfunction, (C) LV cavity dilation, (D) LV wall thickening, and (E) cardiomegaly, even when administered after significant cardiac pathology has already developed. (N=6-12 per group). This data substantiates the efficacy of BET bromodomain inhibition in an experimental setting that is highly relevant to pre-established cardiac disease in humans.

FIG. 12A shows the study design. Mice were subject to permanent proximal left anterior descending artery (LAD) ligation to create a large anterior wall myocardial infarction (MI). JQ1 or vehicle was begun at the indicated doses (25 mg/kg/day or 50 mg/kg/day, intraperitoneal injection) on postoperative day 5. No excess mortality, myocardial rupture, and LV aneurysm formation was seen with JQ1 vs. vehicle control with this dosing regimen. JQ1 attenuates the development of (B) LV systolic dysfunction, (C) LV cavity dilation, (D) LV wall thickening, and (E) cardiomegaly after a large anterior wall myocardial infarction. (N=5 per group). This data substantiates the efficacy of BET bromodomain inhibition in an experimental setting that is highly relevant to human disease.

FIG. 13 shows that BET bromodomains inhibition with JQ1 blocks Doxo induced cardiotoxicity in cultured cardiomyocytes. Neonatal rat ventricular cardiomyocytes (NRVM) were treated with or without JQ1 (250 nM) for 3 hours, followed by treatment±Doxo (1 μM) for another 24 hours. Cells were assayed for apoptosis by TUNEL staining and nuclei were counterstained with DAPI. Images were taken on a fluorescent microscope and TUNEL positive nuclei were quantified (n=5; * p<0.05 vs. vehicle, (−) Doxo; #p<0.05 vs. vehicle, (+) Doxo. These data support that BET bromodomain inhibition with JQ1 can protect the heart from cardiotoxic chemicals such as anthracyclines. These data support the utility of JQ1 as a cardioprotective agent during cancer therapy with the added benefit that JQ1 also has anti-cancer properties.

FIG. 14 shows that JQ1 inhibits cardinal features of pathologic smooth muscle cell activation. All experiments were performed with primary Rat Aortic Smooth Muscle Cells (RASMC), PDGF-bb (10 ng/mL), and JQ1 (500 nM). JQ1 blocks hallmark features of pathologic smooth muscle activation in response to the agonist PDGF-bb such as (A) proliferation (quantified by radiolabeled thymidine incorporation), (B) migration (quantified using a Transwell migration assay), and (C) pathologic gene induction (qRT-PCR shown for Ptgs2/Cox2). These findings support the efficacy of BET bromodomain inhibition against pathologic smooth muscle growth (n=3-6 per group; *p<0.05 vs. vehicle; **p<0.05 vs. PDGF-bb).

FIG. 15 demonstrate efficacy of BET bromodomain inhibition (using JQ1) in pathologic cardiac remodeling in a mouse model of myocardial infarction (MI). (A) Study design. Mice were subject to permanent proximal LAD ligation to create a large anterior wall myocardial infarction (MI). JQ1 or vehicle was begun at the indicated doses (25 mg/kg/day or 50 mg/kg/day, intraperitoneal injection) on postoperative day 5. No excess mortality, myocardial rupture, and LV aneurysm formation was seen with JQ1 vs. vehicle control with this dosing regimen. JQ1 attenuates the development of (B) LV systolic dysfunction, (C) LV cavity dilation, (D) LV wall thickening, and (E) cardiomegaly after a large anterior wall myocardial infarction. (N=5 in sham group, N=10 in MI group).

### DETAILED DESCRIPTION OF THE INVENTION

The present invention is based, at least in part, on the surprising discovery that bromodomain and extraterminal (BET) family of bromodomain-containing proteins (BRD2, BRD3, BRD4 and BRDT) are critical effectors of pathologic cardiac remodeling via their ability to co-activate a broad, but defined stress-induced transcriptional program in the heart. The inventors of the instant application have shown that in vivo BET bromodomain inhibition with the small molecule probe JQ1 potently suppresses pathologic cardiac remodeling and preserves contractile function during exposure to both hemodynamic and neurohormonal stress. Accordingly, aspects of the invention include methods of treating cardiac hypertrophy. The methods comprise administering to a subject in need of such treatment an effective amount of a compound of the invention, e.g., JQ1, to treat cardiac hypertrophy.

Cardiomyopathy (literally “heart muscle disease”) is the measurable deterioration of the function of the myocardium (the heart muscle) for any reason, usually leading to heart failure; common symptoms are dyspnea (breathlessness) and peripheral edema (swelling of the legs). Examples of cardiomyopathy that are independent of inflammation or atherosclerosis are due to chronic hypertension, valvular heart disease (aortic valve stenosis, aortic valve insufficiency, mitral valve insufficiency), peripartum cardiomyopathy, or cardiomyopathy due to genetic mutations (includes familial hypertrophic cardiomyopathy and familial dilated cardiomyopathy). In some embodiments, the cadiomyopathy is cardiac hypertrophy.

As used herein, “cardiac hypertrophy” refers to an enlargement of heart that is activated by stressors such as mechanical and hormonal stimuli and enables the heart to adapt to demands for increased cardiac output or to injury (Morgan and Baker, Circulation 83, 13-25 (1991)). It is the presence of increased cardiac mass. It is typically detected by noninvasive methods such as electrocardiography or imaging modalities such as chest X-ray, cardiac ultrasound (echocardiography), cardiac CT scanning, or cardiac MRI scanning. There are strict clinically defined measurements based on these image modalities. It frequently occurs independently of coronary artery disease or inflammation. Even when present in the asymptomatic state, its presence is strongly associated with adverse future events. There are no currently prescribed therapies for asymptomatic cardiac hypertrophy other than standard treatment of hypertension, if present. Cardiac hypertrophy is physiologically evident in many patients and is largely unrelated to inflammation.

In some embodiments, cardiac hypertrophy can also be evident independent of heart failure, obstructive coronary artery disease, and/or atherosclerosis. As used herein, “heart failure” is a disease that occurs when the heart is unable to maintain organ perfusion at a level sufficient to meet tissue demand, and results in fatigue, breathlessness, multi-organ dysfunction, and early death. Heart failure includes a wide range of disease states such as congestive heart failure, myocardial infarction, tachyarrhythmia, familial hypertrophic cardiomyopathy, ischemic heart disease, idiopathic dilated cardiomyopathy, myocarditis and the like. Heart failure can be caused by any number of factors, including, without limitation, ischemic, congenital, rheumatic, viral, toxic or idiopathic forms. Chronic cardiac hypertrophy is a significantly diseased state which is a precursor to congestive heart failure and cardiac arrest.

“Obstructive coronary artery disease” refers to diseases of the arterial cardiovasculature arising from obstruction of one or more of the coronary arteries. Such diseases include, without limitation, atherosclerosis, thrombosis, restenosis, myocardial infarction, and/or ischemia (including recurrent ischemia) of the coronary arterial vasculature. A symptom of one or more of these diseases may include angina, such as exercise-induced angina, variant angina, stable angina and unstable angina.

“Atherosclerosis” refers to a disorder characterized by the deposition of plaques containing cholesterol and lipids on the innermost layer of the walls of large and medium-sized arteries. Atherosclerosis can also be characterized as a chronic inflammatory disease in which the presence of LDL particles in the vascular wall leads to recruitment of monocytes from the blood, their transformation into macrophages and a dynamic but ultimately unsuccessful attempt to eliminate the LDL particles by phagocytosis. Both the innate and the adaptive immune system appear to contribute to the development of the lesions, and as in many other inflammatory diseases, activation of complement appears to mediate at least part of the tissue damage.

“Atherosclerotic coronary artery disease” refers to the presence of a flow-limiting stenosis detected on coronary angiography (>70% obstruction of luminal diameter) with clinical evidence of reduced myocardial blood flow (symptoms of angina or a positive cardiac stress test).

The subject is an animal, typically a mammal. In one aspect, the subject is a dog, a cat, a horse, a sheep, a goat, a cow or a rodent. In important embodiments, the subject is a human.

In some embodiments, the subject does not have heart failure. In some embodiments, the subject is free of symptoms of obstructive coronary artery disease, including but not limited to angina, such as exercise-induced angina, variant angina, stable angina and unstable angina. In some embodiments, the subject is not being treated for atherosclerosis. For example, the subject is not being treated with statins, anti-platelet medications, beta blocker medications, angiotension-converting enzyme (ACE) inhibitors and calcium channel blockers. In some embodiments, the subject is not being treated for atherosclerosis, as evidenced by an angiogram showing.

In some embodiments, the subject does not have heart failure or atherosclerosis and is not recovering from a myocardial infarction. Acute myocardial infarction (AMI) is the death or necrosis of myocardial cells, caused by the interruption of the blood supply to the heart. The terms “myocardial infarction” and “heart attack” are used herein as having very similar meanings, i.e., the same meanings used by those skilled in the general medical and cardiology fields.

In some embodiments, the subject is over the age of 60 years, and is at risk of developing hypertrophy but is currently asymptomatic. Such subjects can be identified for treatment based on an angiogram.

In some embodiments, the subject is receiving therapy for reducing blood pressure, such as antihypertensive agents. There are many classes of antihypertensives, which lower blood pressure by different means; among the most important and most widely used are the thiazide diuretics, the ACE inhibitors, the calcium channel blockers, the beta blockers, and the angiotensin II receptor antagonists or ARBs. Examples of antihypertensives include, but are not limited to indapamide, chlorthalidone, metolazone, captopril, enalapril, fosinopril, lisinopril, perindopril, quinapril, ramipril, trandolapril, benazepril, amlodipine, Cilnidipine, felodipine, isradipine, lercanidipine, nicardipine, nifedipine, nimodipine, nitrendipine, atenolol, metoprolol, nadolol, nebivolol, oxprenolol, pindolol, propranolol, timolol, candesartan, eprosartan, irbesartan, losartan, olmesartan, telmisartan and valsartan.

Some aspects of the invention involve methods of treating heart failure not arising from inflammation. The method involves administering to a subject in need of such treatment an effective amount of a compound of the invention, e.g., JQ1, to treat the heart failure.

Heart failure not arising from inflammation is heart failure for which an anti-inflammatory medication is not indicated. Thus, subjects having heart failure not arising from inflammation are not administered anti-inflammatory drugs such as but not limited to steroidal, and non-steroidal anti-inflammatory drugs. Heart failure not arising from inflammation is not caused by atherosclerosis, myocardial infarction, and obstructive coronary artery disease.

Typically, the subject does not have obstructive coronary artery disease, as evidenced by an angiogram showing. In some embodiments, the subject is not recovering from a myocardial infarction. In some embodiments, the heart failure is due to:

(i) Heart failure with preserved ejection fraction (HFpEF) without evidence of obstructive coronary artery disease;

(ii) Heat failure due to toxicity of drugs (including anti-cancer agents and drugs of abuse);

(iii) Heart failure caused by ethanol abuse;

(iv) Heart failure due to chronic tachycardia (rapid heart rate);

(v) Heart failure due to endocrine abnormalities (excessive thyroid hormone, growth hormone, diabetes, pheochromocytoma);

(vi) High-output heart failure (includes that which is caused by anemia or peripheral atriovenous shunting);

(vii) Heart failure caused by nutritional deficiencies (including thiamine, selenium, calcium, and magnesium deficiency);

(viii) Heart failure due to viral infection (including HIV) or

(ix) Heart failure due to congenital heart malformations.

In some embodiments, the subject is receiving therapy for reducing blood pressure, such as antihypertensive agents. There are many classes of antihypertensives, which lower blood pressure by different means; among the most important and most widely used are the thiazide diuretics, the ACE inhibitors, the calcium channel blockers, the beta blockers, and the angiotensin II receptor antagonists or ARBs. Examples of antihypertensives include, but are not limited to indapamide, chlorthalidone, metolazone, captopril, enalapril, fosinopril, lisinopril, perindopril, quinapril, ramipril, trandolapril, benazepril, amlodipine, Cilnidipine, felodipine, isradipine, lercanidipine, nicardipine, nifedipine, nimodipine, nitrendipine, atenolol, metoprolol, nadolol, nebivolol, oxprenolol, pindolol, propranolol, timolol, candesartan, eprosartan, irbesartan, losartan, olmesartan, telmisartan and valsartan.

The subject is an animal, typically a mammal. In one aspect, the subject is a dog, a cat, a horse, a sheep, a goat, a cow or a rodent. In important embodiments, the subject is a human.

Some aspects of the invention involve methods for treating myocardial infarction. The method comprises administering to a subject in need of such treatment a compound of the invention, e.g., JQ1, in an amount effective to treat the myocardial infarction. Administration of the compound of the invention, e.g., JQ1 is initiated not sooner than 5 days after the myocardial infarction. In some embodiments, administration of the compound of the invention, e.g., JQ1 is initiated not sooner than 6 days after the myocardial infarction. In some embodiments, administration of the compound of the invention, e.g., JQ1 is initiated not sooner than 7 days after the myocardial infarction. In some embodiments, administration of the compound of the invention, e.g., JQ1 is initiated not sooner than 8, 9, 10, 11, 12, 13, or 14 days after the myocardial infarction.

Typically, the subject is receiving beta blocker and ACE inhibitor treatment. Examples of beta blockers include but are not limited to atenolol, metoprolol, nadolol, nebivolol, oxprenolol, pindolol, propranolol, and timolol. Examples of ACE inhibitors include but are not limited to captopril, enalapril, fosinopril, lisinopril, perindopril, quinapril, ramipril, trandolapril, and benazepril. In some embodiments, the subject does not have atherosclerosis, as evidenced by an angiogram showing. In some embodiments, the subject does not have heart failure.

According to another aspect of the invention, a method for cardioprotection is provided. The method involves administering to a subject receiving a therapy that is cardio toxic a BET inhibitor in an amount effective to inhibit cardio toxicity by such therapy.

It is known in the art that many anti-cancer agents have cardiotoxic effects. Many therapies used to treat cancer, such as but not limited to classic chemotherapeutic agents, monoclonal antibodies that target tyrosine kinase receptors, small molecule tyrosine kinase inhibitors, and even anti-angiogenic drugs and chemoprevention agents such as cyclooxygenase-2 inhibitors, all affect the cardiovascular system. Well-known examples of chemotherapeutic drugs with cardiotoxicity include but are not limited to, anthracyclines, such as Doxorubicin and Daunorubicin, the monoclonal antibody, trastuzumab, 5-fluorouracil, mitoxantrone, paclitaxel or vinca alkaloids, tamoxifen, cyclophosphamide, imatinib, trastuzumab, antimetabolite agents, such as capecitabine or cytarabine, tyrosine kinase inhibitors (TKIs) sorafenib and sunitinib, and the anti-vascular endothelial growth factor antibody bevacizumab.

It has been discovered unexpectedly that BET (bromodomain and extraterminal family of bromodomain-containing proteins (BRD2, BRD3, BRD4, and BRDT)) inhibitors are protective of muscle cell stress. In some embodiments, the BET inhibitors are protective of smooth muscle cell stress. Therefore, the BET inhibitors in general would be useful in protecting a subject against cardiotoxic effects of such anti-cancer molecules.

A BET inhibitor inhibits the binding of BET family bromodomains to acetylated lysine residues. By “BET family bromodomains” is meant a polypeptide comprising two bromodomains and an extraterminal (ET) domain or a fragment thereof having transcriptional regulatory activity or acetylated lysine binding activity. Exemplary BET family members include BRD2, BRD3, BRD4 and BRDT (see WO 2011/143669, incorporated by reference herein). Examples of BET inhibitors include but are not limited to the compounds of the instant invention. Other examples of BET inhibitors can be found, for example, in WO 2011/054843, WO 2009/084693, and JP2008-156311 (each of which is incorporated by reference herein).

According to some aspects of the invention, a method for inhibiting restenosis is provided. The method comprises administering to a subject undergoing an angioplasty and/or receiving a stent a BET inhibitor in an amount effective to inhibit restenosis.

When a major artery is acutely occluded, the results can be serious, such as, for example, infarction of heart muscle. Often times, vascular intervention, including angioplasty, stenting, atherectomy and grafting is often complicated by endothelial and smooth muscle cell proliferation resulting in restenosis or re-clogging of the artery. This may be due to endothelial cell injury caused by the treatment itself. Percutaneous transluminal intervention (PTI), such as stenting, may actually trigger release of fluids and/or solids from a vulnerable plaque into the blood stream, thereby potentially causing a coronary thrombotic occlusion. Therefore, there is a need for the treatment of vulnerable plaques and restenosis.

Various therapies have been attempted for treating or preventing restenosis. For example, the use of stents coated with therapeutic agents has been proposed to help minimize the possibility of restenosis. Stents coated with paclitaxel have been shown to reduce restenosis rates when compared with uncoated stents. The inventors of the present application have discovered that BET inhibitors, such as JQ1, can surprisingly, inhibit muscle cell growth (e.g., smooth muscle cell growth) in connection with ventricular hypertrophy and blood vessel injury. Thus, methods for inhibiting restenosis using BET inhibitors are provided.

As used herein, “restenosis” refers to a renarrowing of a vessel (or other structure) after a procedure performed to relieve a narrowing. The invention aims, in some instances, to reduce the occurrence (or incidence) of restenosis in a subject, and/or to reduce the severity or degree of the restenosis, and/or to reduce or ameliorate the symptoms associated with restenosis. A reduction in the severity or degree of restenosis may be measured directly or indirectly. For example, the severity or degree of restenosis may be measured directly through, for example, measurement of a vessel diameter. Indirect measurements may include functional measurements. The nature of the functional measurement will depend upon the nature and normal function of the damaged vessel. An example of a functional measurement is flow rate and flow quality through the vessel. These measurements are preferably made when the restenosis is likely to occur, based on historical data from comparable but untreated subjects. Such timing may be days, weeks, months or years following treatment. Analysis of symptoms relating to restenosis will also depend on the nature of the vessel(s) that may restenose. If restenosis may occur in the vasculature, then symptoms include any cardiovascular symptoms relating to blood flow impairment, including but not limited to cardiac and cerebral symptoms. These may include chest pain (angina), particularly following physical exertion, unusual fatigue, shortness of breath, and chest pressure. Biological markers may also be measured as an indicator of restenosis. An example of a biological marker is troponin, which is elevated in the presence of restenosis. Various tests are available to detect restenosis including imaging tests (e.g., CT, magnetic resonance imaging, radionuclide imaging, angiogram, Doppler ultrasound, MRA, etc.), and functional tests such as an exercise stress test.

Typically, the subject is undergoing angioplasty. The term “angioplasty” includes the alteration of the structure of a vessel, either by dilating the vessel using a balloon inside the lumen or by other surgical procedure. The term “angioplasty” includes percutaneous transluminal coronary angioplasty. In some embodiments, the subject is receiving a stent. Stents are tubular scaffold structures used to prop open blood vessels and other body lumens. The most widespread use of stents is to open clogged coronary arteries and prevent restenosis.

In some embodiments, the BET inhibitor is administered locally at the site of a stenosis. A stenosis is an abnormal narrowing in a blood vessel or other tubular organ or structure. In some embodiments, the BET inhibitor is administered via a catheter. In some embodiments, the BET inhibitor is administered as an element of a coating on a stent.

A BET inhibitor inhibits the binding of BET family bromodomains to acetylated lysine residues. By “BET family bromodomains” is meant a polypeptide comprising two bromodomains and an extraterminal (ET) domain or a fragment thereof having transcriptional regulatory activity or acetylated lysine binding activity. Exemplary BET family members include BRD2, BRD3, BRD4 and BRDT (see WO 2011/143669, incorporated by reference herein). Examples of BET inhibitors include but are not limited to the compounds of the instant invention. Other examples of BET inhibitors can be found, for example, in WO 2011/054843, WO 2009/084693, and JP2008-156311 (each of which is incorporated by reference herein).

### Compounds of the Invention

The invention provides compounds (e.g., JQ1 and compounds of formulas delineated herein and in WO 2011/143669, incorporated by reference herein) that bind in the binding pocket of the apo crystal structure of the first bromodomain of a BET family member (e.g., BRD4). In certain embodiments, a compound of the invention can bind to a BET family member and reduce the biological activity of the BET family member (e.g., reduce elongation) and/or disrupt the subcellular localization of the BET family member (e.g., reduce chromatin binding).

In certain embodiments, a compound of the invention can prevent, inhibit, or disrupt, or reduce by at least 10%, 25%, 50%, 75%, or 100% the biological activity of a BET family member (e.g., BRD2, BRD3, BRD4, BRDT) and/or disrupt the subcellular localization of such proteins, e.g., by binding to a binding site in a bromodomain apo binding pocket.

In certain embodiments, a compound of the invention is a small molecule having a molecular weight less than about 1000 daltons, less than 800, less than 600, less than 500, less than 400, or less than about 300 daltons. Examples of compounds of the invention include JQ1 and other compounds that bind the binding pocket of the apo crystal structure of the first bromodomain of a BET family member (e.g., BRD4 (hereafter referred to as BRD4(1); PDB ID 20SS). JQ1 is a novel thieno-triazolo-1,4-diazepine. The invention further provides pharmaceutically acceptable salts of such compounds.

In one aspect, the invention provides a compound of Formula I:

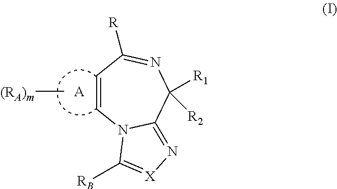

wherein

X is N or CR₅;

R₅ is H, alkyl, cycloalkyl, heterocycloalkyl, aryl, or heteroaryl, each of which is optionally substituted;

R_{B} is H, alkyl, hydroxylalkyl, aminoalkyl, alkoxyalkyl, haloalkyl, hydroxy, alkoxy, or —COO—R3, each of which is optionally substituted;

ring A is aryl or heteroaryl;

each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;

R is alkyl, cycloalkyl, heterocycloalkyl, aryl, or heteroaryl; each of which is optionally substituted;

Ri is —(CH₂)ₙ-L, in which n is 0-3 and L is H, —COO—R3, —CO—R3, —CO—N(R₃R₄), —S(0)₂-R₃, —S(0)₂—N(R₃R₄), N(R₃R4), N(R4)C(0)R₃, optionally substituted aryl, or optionally substituted heteroaryl;

R₂ is H, D (deuterium), halogen, or optionally substituted alkyl;

each R₃ is independently selected from the group consisting of:

(i) H, aryl, substituted aryl, heteroaryl, or substituted heteroaryl;

(ii) heterocycloalkyl or substituted heterocycloalkyl;

(iii) —C₁-C₈ alkyl, —C₂-C₈ alkenyl or —C₂-C₈ alkynyl, each containing 0, 1, 2, or 3 heteroatoms selected from O, S, or N; —C₃-Ci₂ cycloalkyl, substituted —C₃-Ci₂ cycloalkyl, —C₃—Ci₂ cycloalkenyl, or substituted —C₃-Ci₂ cycloalkenyl, each of which may be optionally substituted; and

(iv) NH₂, N═CR₄R₆;

each R₄ is independently H, alkyl, alkyl, cycloalkyl, heterocycloalkyl, aryl, or heteroaryl, each of which is optionally substituted;

or R₃ and R₄ are taken together with the nitrogen atom to which they are attached to form a 4-10-membered ring;

R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;

m is 0, 1, 2, or 3;

provided that

(a) if ring A is thienyl, X is N, R is phenyl or substituted phenyl, R₂ is H, RB is methyl, and Ri is —(CH₂)ₙ-L, in which n is 1 and L is —CO—N(R₃R₄), then R₃ and R₄ are not taken together with the nitrogen atom to which they are attached to form a morpholino ring;

(b) if ring A is thienyl, X is N, R is substituted phenyl, R₂ is H, RB is

methyl, and Ri is —(CH₂)ₙ-L, in which n is 1 and L is —CO—N(R₃R₄), and one of R₃ and R₄ is H, then the other of R₃ and R₄ is not methyl, hydroxyethyl, alkoxy, phenyl, substituted phenyl, pyridyl or substituted pyridyl; and

(c) if ring A is thienyl, X is N, R is substituted phenyl, R₂ is H, R_{B} is

methyl, and Ri is —(CH₂)ₙ-L, in which n is 1 and L is —COO—R₃, then R₃ is not methyl or ethyl;

or a salt, solvate or hydrate thereof.

In certain embodiments, R is aryl or heteroaryl, each of which is optionally substituted.

In certain embodiments, L is H, —COO—R₃, —CO—N(R₃R₄), —S(0)₂—R₃, —S(0)₂—N(R₃R₄), N(R₃R₄), N(R₄)C(0)R₃ or optionally substituted aryl. In certain embodiments, each R₃ is independently selected from the group consisting of: H, —C₁-C₈ alkyl, which is optionally substituted, containing 0, 1, 2, or 3 heteroatoms selected from O, S, or N; or NH₂, N═CR₄R₆.

In certain embodiments, R₂ is H, D, halogen or methyl.

In certain embodiments, R_{B} is alkyl, hydroxyalkyl, haloalkyl, or alkoxy; each of which is optionally substituted.

In certain embodiments, R_{B} is methyl, ethyl, hydroxy methyl, methoxymethyl, trifluoromethyl, COOH, COOMe, COOEt, or COOCH₂OC(0)CH₃.

In certain embodiments, ring A is a 5 or 6-membered aryl or heteroaryl. In certain embodiments, ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In certain embodiments, ring A is phenyl or thienyl.

In certain embodiments, m is 1 or 2, and at least one occurrence of R_{A} is methyl.

In certain embodiments, each R_{A} is independently H, an optionally substituted alkyl, or any two R_{A} together with the atoms to which each is attached, can form an aryl.

In another aspect, the invention provides a compound of Formula II:

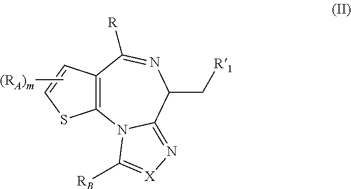

wherein

X is N or CR₅;

R₅ is H, alkyl, cycloalkyl, heterocycloalkyl, aryl, or heteroaryl, each of which is optionally substituted;

R_{B} is H, alkyl, hydroxylalkyl, aminoalkyl, alkoxyalkyl, haloalkyl, hydroxy, alkoxy, or —COO—R₃, each of which is optionally substituted;

each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;

R is alkyl, cycloalkyl, heterocycloalkyl, aryl, or heteroaryl, each of which is optionally substituted;

R′i is H, —COO—R₃, —CO—R₃, optionally substituted aryl, or optionally substituted heteroaryl;

each R₃ is independently selected from the group consisting of:

(i) H, aryl, substituted aryl, heteroaryl, substituted heteroaryl;

(ii) heterocycloalkyl or substituted heterocycloalkyl;

(iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl; —C₃-C₁₂ cycloalkenyl, or substituted —C₃-C₁₂cycloalkenyl; each of which may be optionally substituted;

m is 0, 1, 2, or 3;

provided that if R′₁ is —COO—R₃, X is N, R is substituted phenyl, and R_{B} is methyl, then R₃ is not methyl or ethyl;

or a salt, solvate or hydrate thereof.

In certain embodiments, R is aryl or heteroaryl, each of which is optionally substituted. In certain embodiments, R is phenyl or pyridyl, each of which is optionally substituted. In certain embodiments, R is p-Cl-phenyl, o-Cl-phenyl, m-Cl-phenyl, p-F-phenyl, o-F-phenyl, m-F-phenyl or pyridinyl.

In certain embodiments, R′i is —COO—R₃, optionally substituted aryl, or optionally substituted heteroaryl; and R₃ is —C₁-C₈ alkyl, which contains 0, 1, 2, or 3 heteroatoms selected from O, S, or N, and which may be optionally substituted. In certain embodiments, R′i is —COO—R₃, and R₃ is methyl, ethyl, propyl, i-propyl, butyl, sec-butyl, or t-butyl; or R′i is H or optionally substituted phenyl.

In certain embodiments, R_{B} is methyl, ethyl, hydroxy methyl, methoxymethyl, trifluoromethyl, COOH, COOMe, COOEt, COOCH₂OC(0)CH₃.

In certain embodiments, R_{B} is methyl, ethyl, hydroxy methyl, methoxymethyl, trifluoromethyl, COOH, COOMe, COOEt, or COOCH₂OC(0)CH₃.

In certain embodiments, each R_{A} is independently an optionally substituted alkyl, or any two R_{A} together with the atoms to which each is attached, can form a fused aryl.

In certain embodiments, each R_{A} is methyl.

In another aspect, the invention provides a compound of formula III:

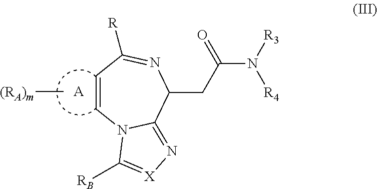

wherein

X is N or CR₅;

R₅ is H, alkyl, cycloalkyl, heterocycloalkyl, aryl, or heteroaryl, each of which is optionally substituted;

RB is H, alkyl, hydroxylalkyl, aminoalkyl, alkoxyalkyl, haloalkyl, hydroxy, alkoxy, or —COO—R3, each of which is optionally substituted;

ring A is aryl or heteroaryl;

each R_{A} is independently alkyl, cycloalkyl, heterocycloalkyl, aryl, or heteroaryl, each of which is optionally substituted; or any two RA together with the atoms to which each is attached, can form a fused aryl or heteroaryl group;

R is alkyl, cycloalkyl, heterocycloalkyl, aryl, or heteroaryl, each of which is optionally substituted;

each R₃ is independently selected from the group consisting of:

(i) H, aryl, substituted aryl, heteroaryl, or substituted heteroaryl;

(ii) heterocycloalkyl or substituted heterocycloalkyl;

(iii) —C₁-C₈ alkyl, —C₂-C₈ alkenyl or —C₂-C₈ alkynyl, each containing 0, 1, 2, or 3 heteroatoms selected from O, S, or N; —C₃-Ci₂ cycloalkyl, substituted —C₃-Ci₂ cycloalkyl, —C₃-Ci₂ cycloalkenyl, or substituted —C₃-Ci₂ cycloalkenyl, each of which may be optionally substituted; and

(iv) NH₂, N═CR₄R₆;

each R₄ is independently H, alkyl, alkyl, cycloalkyl, heterocycloalkyl, aryl, or heteroaryl, each of which is optionally substituted;

or R₃ and R₄ are taken together with the nitrogen atom to which they are attached to form a 4-10-membered ring;

R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;

m is 0, 1, 2, or 3;

provided that:

(a) if ring A is thienyl, X is N, R is phenyl or substituted phenyl, R_{B} is methyl, then R₃ and R₄ are not taken together with the nitrogen atom to which they are attached to form a morpholino ring; and

(b) if ring A is thienyl, X is N, R is substituted phenyl, R₂ is H, R_{B} is methyl, and one of R₃ and R₄ is H, then the other of R₃ and R₄ is not methyl, hydroxyethyl, alkoxy, phenyl, substituted phenyl, pyridyl or substituted pyridyl;

or a salt, solvate or hydrate thereof.

In certain embodiments, R is aryl or heteroaryl, each of which is optionally substituted. In certain embodiments, R is phenyl or pyridyl, each of which is optionally substituted.

In certain embodiments, R is p-Cl-phenyl, o-Cl-phenyl, m-Cl-phenyl, p-F-phenyl, o-F-phenyl, m-F-phenyl or pyridinyl. In certain embodiments, R₃ is H, NH₂, or N═CR₄R₆.

In certain embodiments, each R₄ is independently H, alkyl, cycloalkyl, heterocycloalkyl, aryl, heteroaryl; each of which is optionally substituted.

In certain embodiments, R₆ is alkyl, alkenyl, cycloalkyl, cycloalkenyl, heterocycloalkyl, aryl, or heteroaryl, each of which is optionally substituted.

In another aspect, the invention provides a compound of formula IV:

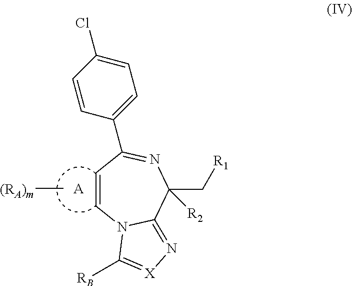

wherein

X is N or CR₅;

R₅ is H, alkyl, cycloalkyl, heterocycloalkyl, aryl, or heteroaryl, each of which is optionally substituted;

R_{B} is H, alkyl, hydroxylalkyl, aminoalkyl, alkoxyalkyl, haloalkyl, hydroxy, alkoxy, or —COO—R3, each of which is optionally substituted;

ring A is aryl or heteroaryl;

each R_{A} is independently alkyl, cycloalkyl, heterocycloalkyl, aryl, or heteroaryl, each of which is optionally substituted; or any two R_{A} together with the atoms to which each is attached, can form a fused aryl or heteroaryl group;

Ri is —(CH₂)ₙ-L, in which n is 0-3 and L is H, —COO—R3, —CO—R3, —CO—N(R₃R₄), —S(0)₂-R₃, —S(0)₂-N(R₃R₄), N(R₃R4), N(R4)C(0)R₃, optionally substituted aryl, or optionally substituted heteroaryl;

R₂ is H, D, halogen, or optionally substituted alkyl;

each R₃ is independently selected from the group consisting of:

(i) H, aryl, substituted aryl, heteroaryl, or substituted heteroaryl;

(ii) heterocycloalkyl or substituted heterocycloalkyl;

(iii) —C₁-C₈ alkyl, —C₂-C₈ alkenyl or —C₂-C₈ alkynyl, each containing 0, 1, 2, or 3 heteroatoms selected from O, S, or N; —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted; and

(iv) NH₂, N═CR₄R₆;

each R₄ is independently H, alkyl, alkyl, cycloalkyl, heterocycloalkyl, aryl, or heteroaryl, each of which is optionally substituted;

or R₃ and R₄ are taken together with the nitrogen atom to which they are attached to form a 4-10-membered ring;

R₆ is alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring;

m is 0, 1, 2, or 3;

provided that

(a) if ring A is thienyl, X is N, R₂ is H, RB is methyl, and Ri is —(CH₂)ₙ-L, in which n is 0 and L is —CO—N(R₃R₄), then R₃ and R₄ are not taken together with the nitrogen atom to which they are attached to form a morpholino ring;

(b) if ring A is thienyl, X is N, R₂ is H, RB is methyl, and Ri is —(CH₂)ₙ-L, in which n is 0 and L is —CO—N(R₃R₄), and one of R₃ and R₄ is H, then the other of R₃ and R₄ is not methyl, hydroxyethyl, alkoxy, phenyl, substituted phenyl, pyridyl or substituted pyridyl; and

(c) if ring A is thienyl, X is N, R₂ is H, R_{B} is methyl, and Ri is —(CH₂)ₙ-L, in which n is 0 and L is —COO—R₃, then R₃ is not methyl or ethyl; or a salt, solvate or hydrate thereof.

In certain embodiments, Ri is —(CH₂)ₙ-L, in which n is 0-3 and L is

—COO—R₃, optionally substituted aryl, or optionally substituted heteroaryl; and R₃ is —C₁-C₈ alkyl, which contains 0, 1, 2, or 3 heteroatoms selected from O, S, or N, and which may be optionally substituted. In certain embodiments, n is 1 or 2 and L is alkyl or —COO—R₃, and R₃ is methyl, ethyl, propyl, i-propyl, butyl, sec-butyl, or t-butyl; or n is 1 or 2 and L is H or optionally substituted phenyl.

In certain embodiments, R₂ is H or methyl.

In certain embodiments, R_{B} is methyl, ethyl, hydroxy methyl, methoxymethyl, trifluoromethyl, COOH, COOMe, COOEt, COOCH₂OC(0)CH₃.

In certain embodiments, ring A is phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In certain embodiments, each R_{A} is independently an optionally substituted alkyl, or any two R_{A} together with the atoms to which each is attached, can form an aryl.

The invention also provides compounds of Formulae V-XXII, and all compounds described in WO 2011/143669 and incorporated by reference herein.

In certain embodiments, the compound is (+)-JQ1:

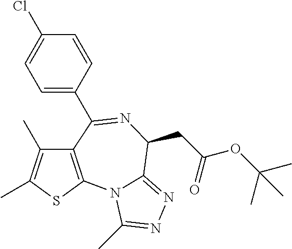 [SMILES: Cc1sc2c(c1C)C(c1ccc(Cl)cc1)=N[C@@H](CC(=O)OC(C)(C)C)c1nnc(C)n1-2]

a salt, solvate or hydrate thereof.

The compounds of the invention are administered in an effective amount. An effective amount is a dose sufficient to provide a medically desirable result and can be determined by one of skill in the art using routine methods. In some embodiments, an effective amount is an amount which results in any improvement in the condition being treated. In some embodiments, an effective amount may depend on the type and extent of the disease or condition being treated and/or use of one or more additional therapeutic agents. However, one of skill in the art can determine appropriate doses and ranges of therapeutic agents to use, for example based on in vitro and/or in vivo testing and/or other knowledge of compound dosages.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several or many days (depending on the mode of administration and the factors discussed above).

In some embodiments, an effective amount is an amount that would halt or inhibit the progression of cardiomyopathy and/or cardiac hypertrophy. In some embodiments, an effective amount is an amount that would even delay the onset of cardiomyopathy and/or hypertrophy in a subject having risk factors for cardiomyopathy and/or hypertrophy.

In some embodiments, an effective amount is an amount that would halt or inhibit the progression of heart failure. In some embodiments, as effective amount is an amount that would even delay the onset of heart failure in a subject having risk factors for heart failure.

In some embodiments, an effective amount is the amount of a BET inhibitor that would prevent and/or reduce injury of heart. The specific therapeutically effective dose level for any particular patient will depend upon a variety of factors including the severity of the condition; activity of the specific compound employed; the specific composition employed and the age of the subject. The person responsible for administration will, in any event, determine the appropriate dose for the individual subject.

In some embodiments, an effective amount is an amount of a BET inhibitor that is sufficient to inhibit or halt proliferation of coronary smooth muscle cells at the site of vascular injury following angioplasty. The amount of BET inhibitor which constitutes an “effective amount” will vary depending on the BET inhibitor used, the severity of the restenosis, and the age and body weight of the human to be treated, but can be determined routinely by one of ordinary skill in the art having regard to his own knowledge and to this disclosure.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Methods

### Animal Models.

All protocols concerning animal use were approved by the Institutional Animal Care and Use Committee at Case Western Reserve University and conducted in accordance with the NIH Guide for the Care and Use of Laboratory Animals. All models were conducted in C57Bl/6J mice (Jackson Laboratories), which were maintained in a pathogen-free facility with standard light/dark cycling and access to food and water ad libitum.

### Human Samples.

LV samples from healthy human hearts were obtained were obtained as described (Hannenhalli et al., 2006; Margulies et al., 2005) in accordance with the Investigation Review Committee at the Hospital of the University of Pennsylvania, Philadelphia, Pa. Nuclear protein was extracted using the NE-Per kit (Thermo Scientific #78833) according to manufacturer's instructions. Gene expression profiles from left ventricles obtained from non-failing versus failing human hearts were curated from a published dataset (Hannenhalli et al., 2006).

### Preparation of JQ1.

JQ1 was synthesized and purified in the laboratory of Dr. James Bradner (DFCI) as previously published (Filippakopoulos et al., 2010). For in vivo experiments, a stock solution (50 mg/mL in DMSO) was diluted to a working concentration of 5 mg/mL in aqueous carrier (10% hydroxypropyl β-cyclodextrin; Sigma C0926) using vigorous vortexing. Mice were injected at a dose of 50 mg/kg given intraperitoneally once daily. Vehicle controls were given an equal amount of DMSO in carrier solution. All solutions were prepared and administered using sterile technique. For in vitro experiments, JQ1 and other BET inhibitors were dissolved in DMSO and administered to cells at indicated concentrations using an equal volume of DMSO as control. The BET inhibitors used were as follows: iBET, iBET-151, RVX-208, and PF-1.

### Transverse Aortic Constriction and Chronic PE Infusion in Mice.

All mice were C57Bl/6J littermate males aged 10-12 weeks. For TAC, mice were anesthetized with ketamine/xylazine, mechanically ventilated (Harvard apparatus), and subject to thoracotomy. The aortic arch was constricted between the left and right carotid arteries using a 7.0 silk suture and a 27-gauge needle as previously described (Hu et al., 2003). In our hands, this protocol a consistent peak pressure gradient of approximately 50 mmHg across the constricted portion of the aorta. For PE infusion, mice were anesthetized using continuous 1% inhalational isofluorane. Mini-osmotic pumps (Alzet 2004, Durect Corp.) were filled with phenylephrine hydrochloride (PE, Sigma) or vehicle (normal saline) and implanted subcutaneously on the dorsal aspect of the mouse. PE was infused at a dose of 75 mg/kg/day for 17 days. Injections of JQ1 or vehicle were begun 1.5 days postoperatively.

### Echocardiography, Blood Pressure, and Endurance Exercise Capacity Measurements.

For transthoracic echocardiography, mice were anesthetized with 1% inhalational isofluorane and imaged using the Vevo 770 High Resolution Imaging System (Visual Sonics, Inc.) and the RMV-707B 30 MHz probe. Measurements were obtained from M-mode sampling and integrated EKV images taken in the LV short axis at the mid-papillary level (Haldar et al., 2010). Measurements of pressure gradients across the constricted portion of the aorta were obtained by high frequency Doppler as previously described (Liu et al., 2012). Conscious tail-vein systolic blood pressure was measured using the BP2000 Blood Pressure Analysis System (Visitech Systems, Inc.) as recommended by the manufacturer. To allow mice to adapt to the apparatus, we performed daily blood pressure measurements for one week prior to beginning experiments. Treadmill endurance exercise testing was performed on a motorized mouse treadmill (Columbus Instruments) as previously described (Haldar et al., 2012).

### NRVM Culture.

NRVM were isolated from the hearts of 2 day old Sprague-Dawley rat pups (Charles River) and maintained under standard conditions as described (Haldar et al., 2010). The cells were differentially plated for 1.5 h in cell culture dishes to remove contaminating non-myocytes. Unless otherwise stated, NRVM were plated at a density of 10⁵ cells/mL. Cells were initially plated in growth medium (DMEM supplemented with 5% FBS, 100 U/mL penicillin-streptomycin, and 2 mM L-glutamine) for 24-36 hours and maintained in serum-free media thereafter (DMEM supplemented with 0.1% BSA, 1% insulin-transferrin-selenium liquid media supplement (Sigma 13146), 100 U/mL penicillin-streptomycin, and 2 mM L-glutamine). Media was changed every 2-3 days. Prior to stimulation with agonists, NRVM were maintained in serum-free medium for 48-72 hours. For hypertrophic stimulation, NRVM were incubated with JQ1 versus DMSO at indicated concentrations for 6 h followed by stimulation with PE (100 μM) for indicated timepoints.

### NRVM BRD4 Immunofluorescence.

NRVM were grown on glass coverslips in 6-well dishes. Cells were fixed in PBS containing 3% PFA (15 min), permeabilized in PBST/0.25% Triton X-100 (10 min), and blocked in PBST/5% horse serum for 1 h. Primary antibodies (anti sarcomeric α-actinin, Sigma A7811, 1:800; anti-BRD4, Bethyl A301-985A, 1:250) were co-incubated in PBST/5% horse serum for 1 h. Secondary antibodies (donkey α-mouse Alexa 594 red; donkey α-rabbit Alexa 488 green; Jackson Immuno-research) were co-incubated at 1:1000 each in PBST/5% horse serum for 1 h. Coverslips were mounted onto glass slides with mounting media containing DAPI. Images were taken on a fluorescent microscope.

### Cell Area Measurements.

NRVM were plated on glass coverslips in 6-well dishes at a density of 10⁵ cells/mL. After treatments, cells were briefly fixed in PBS containing 2% PFA, permeabilized with PBST/0.1% Triton X-100, and blocked in PBST/5% horse serum. Primary antibody was anti-sarcomeric α-actinin (Sigma A7811) at 1:800. Fluorophore-tagged anti-mouse secondary antibody (α-mouse Alexa 488 green) was used at 1:1000 dilution. Coverslips were mounted on glass slides with mounting media containing DAPI. Quantitation of cardiomyocyte cell surface area was performed on α-actinin-stained cardiomyocytes using fluorescent microscopy and NIH Image J software as previously described (Liang et al., 2001). Analysis consisted of at least 100 cardiomyocytes in 20-30 fields at 400× magnification. This process was replicated in a least three independent experiments, and the data were combined.

RNA Purification and qRT-PCR.

For tissue RNA, a 10-20 mg piece of mouse heart tissue was preserved in RNA Later stabilization reagent (Qiagen) followed by mechanical disruption/homogenization in PureZOL (BioRad) on a TissueLyser (Qiagen) using stainless steel beads (Qiagen). The aqueous phase was extracted with chloroform. RNA was purified from the aqueous phase using the Aurum purification kit (BioRad #732-6830) following manufacturer's instructions. For cellular samples, total RNA from NRVM was isolated using the High Pure RNA isolation kit (Roche #11828665001) with on-column DNAase treatment according to manufacturer's directions. Purified RNA was reverse transcribed to complementary DNA using the iScript™ RT Supermix (Biorad #170-8841) following manufacturer's protocol. Quantitative real-time PCR was performed using TaqMan chemistry (Fast Start Universal Probe Master (Roche cat#4914058001) and labeled probes from the Roche Universal Probe Library System) on a Roche LightCycler. Relative expression was calculated using the AACt-method with normalization to constitutive genes as indicated.

### Western Blotting.

For total cellular protein, cells were lysed in RIPA Buffer (Sigma R0278) supplemented with protease inhibitor tablets (Roche cat#4693132001). Nuclear protein was isolated using the NE-Per Kit (Thermo Scientific #78833) according to manufacturer's instructions. 20-40 μg of whole cell protein extracts or 20 μg of nuclear protein extracts were subject to SDS PAGE, transfer to nitrocellulose membranes, and Western blotting using the following antibodies: BRD4 (Bethyl #A301-985A), tubulin (Sigma T9026), RNA Polymerase II (Santa Cruz N-20, sc-899).

### Brd4 Knockdown.

For shRNA against mouse/rat Brd4, the hairpin sequence

(SEQ ID NO: 1)
5′-GCGGTAAGATGTACATCAA ACGTGTGCTGTCCGT
TTGGTGTACATCTTGCTGC-3′

(loop sequence is underlined) was subcloned into the pEQ adenoviral-shRNA vector (Welgen, Inc.). Recombinant adenoviruses for sh-Brd4 and sh-control (scrambled shRNA) were amplified and purified by Welgen, Inc. NRVM were incubated with adenovirus (5-10 MOI) for 24 hours, followed by replacement of fresh serum-free media for another 24 hours. 48 hours after initial infection, cells were stimulated with PE.

### Chromatin Immunoprecipitation.

NRVM were plated in 15 cm dishes at 5×10⁶ cells/dish. Chromatin pooled from approximately 15×10⁶ NRVM were used for each immunoprecipitation. After indicated treatments, NRVM were fixed directly on the dish with 1% formaldehyde for 10 minutes followed by quenching with 0.125M glycine for 5 minutes. Chromatin was extracted, followed by shearing on a BioRuptor (Diagenode; total 16 cycles, hi-power, 30 sec on/off). The sonicated chromatin was immunoprecipitated with 5 μg of antibody bound to Dynabeads (Invitrogen) followed by extensive washing and elution. Immunoprecipitate and input chromatin samples were then reverse cross-linked followed by purification of genomic DNA. Target and non-target regions of genomic DNA were amplified by qRT-PCR in both the immunoprecipitates and input samples using Sybrgreen chemistry. Enrichment data were analyzed by calculating the immunoprecipitated DNA percentage of input DNA for each sample as previously described (Ott et al., 2012). Antibodies used in ChIP were BRD4 (Bethyl #A301-985A) and RNA Polymerase II (Santa Cruz N-20, sc-899).

### Histological Analysis.

Short-axis heart sections from the mid-ventricle were fixed in PBS/4% paraformaldehyde and embedded in paraffin. Cardiomyocyte cross sectional area was determined by staining with rhodamine-conjugated wheat-germ agglutinin (Vector Laboratories RL-1022) as quantified as previously described (Froese et al., 2011). Fibrosis was visualized using Masson's Trichrome staining kit (Biocare medical) with quantification of fibrotic area as previously described (Song et al., 2010). Terminal deoxynucleotidyl transferase dUTP nick-end label (TUNEL) staining and quantification was performed as previously described (Song et al., 2010) using the ApopTag Plus kit (Millipore) according to manufacturer's instructions. Myocardial capillary staining was performed in frozen LV sections using anti-PECAM-1 antibodies (EMD Millipore cat#CBL-1337) as previously described (Haldar et al., 2010).

### Statistical Analysis.

Data are reported as mean±standard error. The statistical methods used in analysis of microarray data are detailed separately above. Comparison of means between two groups was analyzed using a two-tailed Student's t-test with Bonferroni correction for multiple comparisons. For all analyses, P values<0.05 were considered significant.

### Results:

### BET Bromodomains are Cell-Autonomous Regulators of Pathologic Cardiomyocyte Hypertrophy In Vitro.

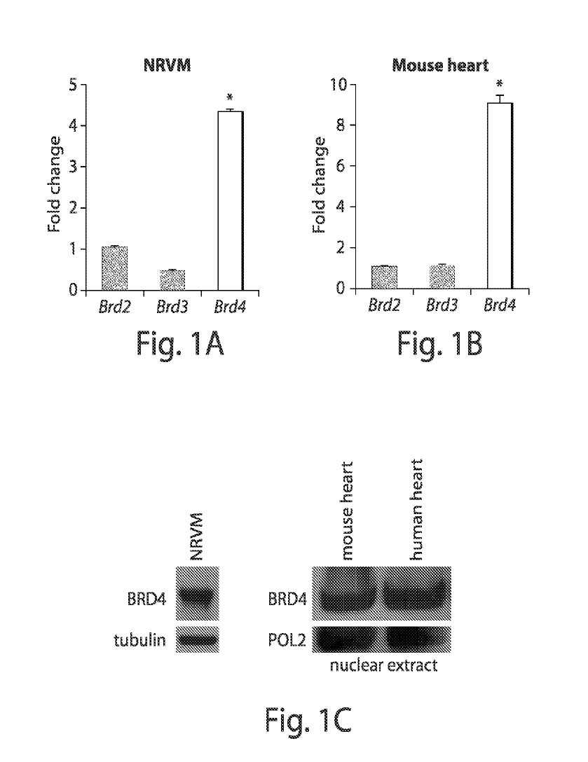

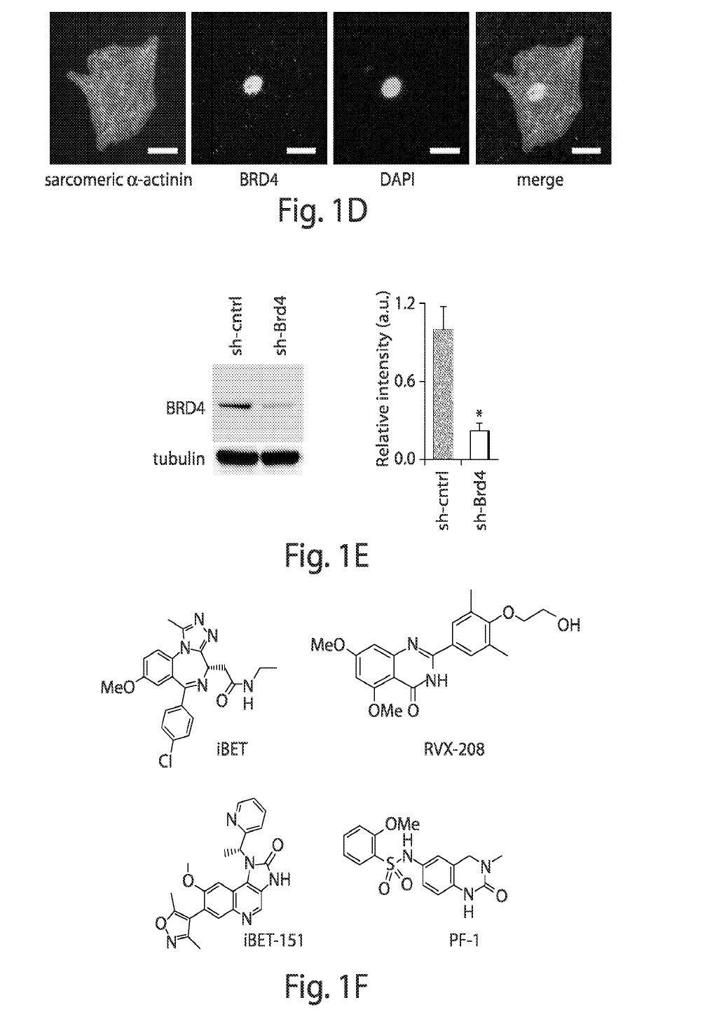

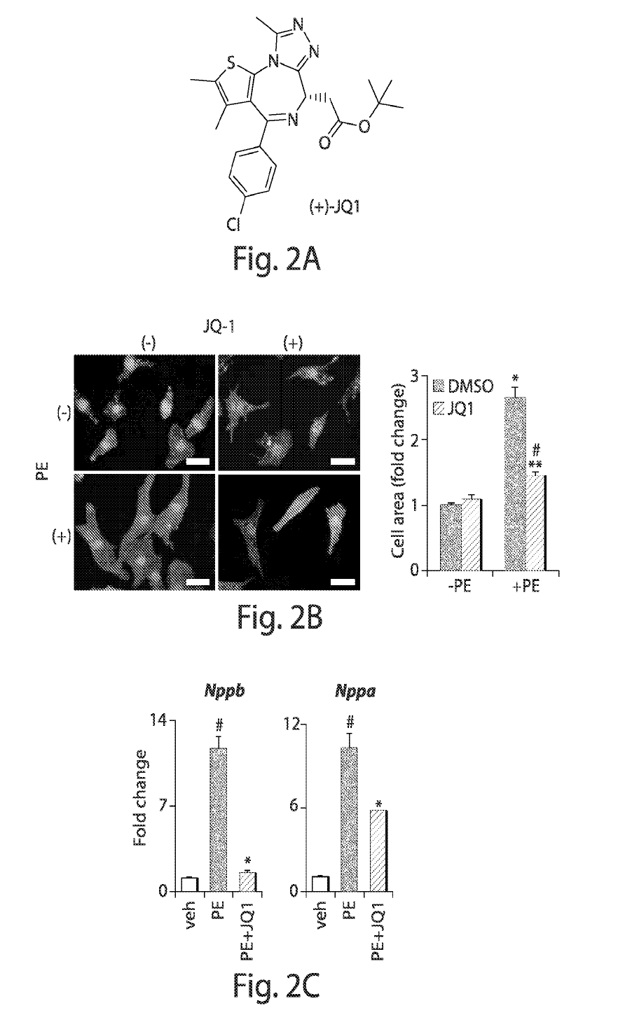

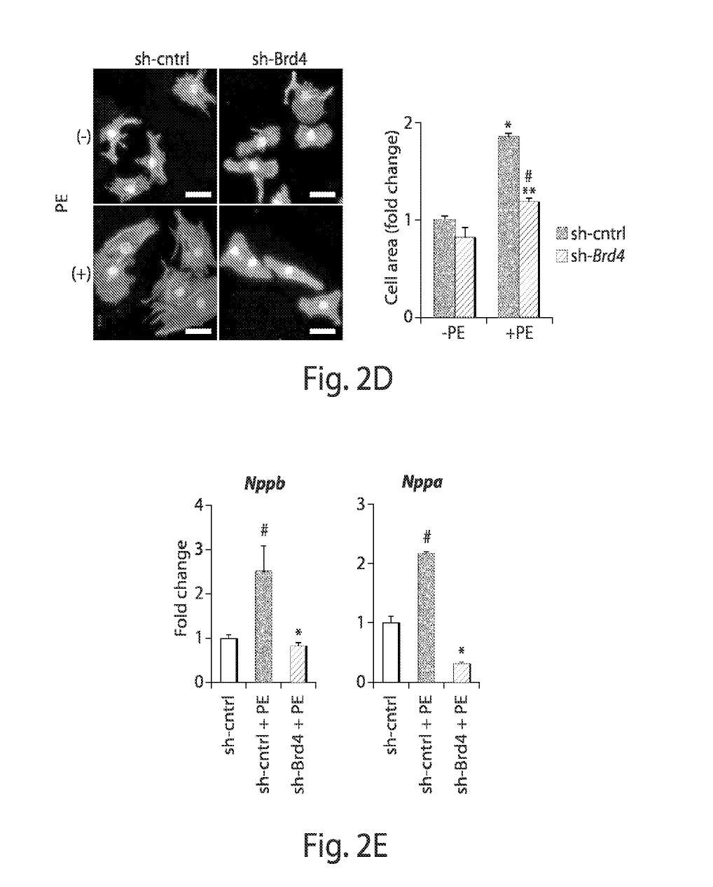

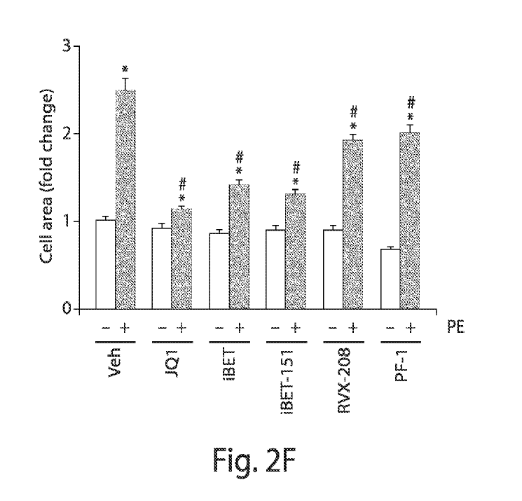

The expression patterns of BETs in the heart was assessed. Analysis of neonatal rat ventricular cardiomyocytes (NRVM) and adult mouse ventricular tissue revealed that Brd2, Brd3, and Brd4 were detectable with Brd4 being the highest expressed transcript (FIG. 1A-B). Western blots in NRVM, mouse heart tissue, and human heart tissue confirmed abundant BRD4 expression (FIG. 1C) and immunofluorescence staining of NRVM demonstrated BRD4 to be nuclear localized (FIG. 1D). As BETs are known to be critical regulators of cellular transformation via their ability to transcriptionally co-activate stimulus and cell-state specific gene expression programs (Filippakopoulos et al., 2010; Lockwood et al., 2012), it was hypothesized that they might play a role in cardiomyocyte hypertrophy. To explore the role of BETs in this process, the properties of the small molecule probe JQ1 was leveraged (FIG. 2A), which specifically and potently inhibits BET function though competitive binding of the second bromodomain and resultant displacement of these epigenetic reader proteins from acetylated chromatin (Filippakopoulos et al., 2010). In the widely used NRVM model (Simpson et al., 1982; Starksen et al., 1986), nanomolar doses of JQ1 significantly blocked phenylephrine (PE) mediated cellular hypertrophy (FIG. 2B) and pathologic gene induction (FIG. 2C). In a similar manner, knockdown of Brd4 in NRVM (FIG. 1E) also attenuated PE mediated hypertrophic growth (FIG. 2D) and pathologic gene induction (FIG. 2E). Next a number of structurally diverse BET inhibitors (iBET, iBET-151, RVX-208, PF-1; FIG. 1F) were assessed for their ability to inhibit cardiomyocyte hypertrophy. At equimolar doses, it was found that inhibition of agonist-induced cardiomyocyte hypertrophy was indeed a class effect of BET inhibitors, with the relative potency of these compounds correlating with their known IC50 against BRD4 (Filippakopoulos et al., 2010). Together, these data demonstrate that BET bromodomain proteins are cell autonomous regulators of pathologic cardiomyocyte hypertrophy and that the small molecule BET inhibitor JQ1 has potent anti-hypertrophic effects in vitro.

### BETs are Required for Induction of a Pathologic Gene Expression Program in Cardiomyocytes

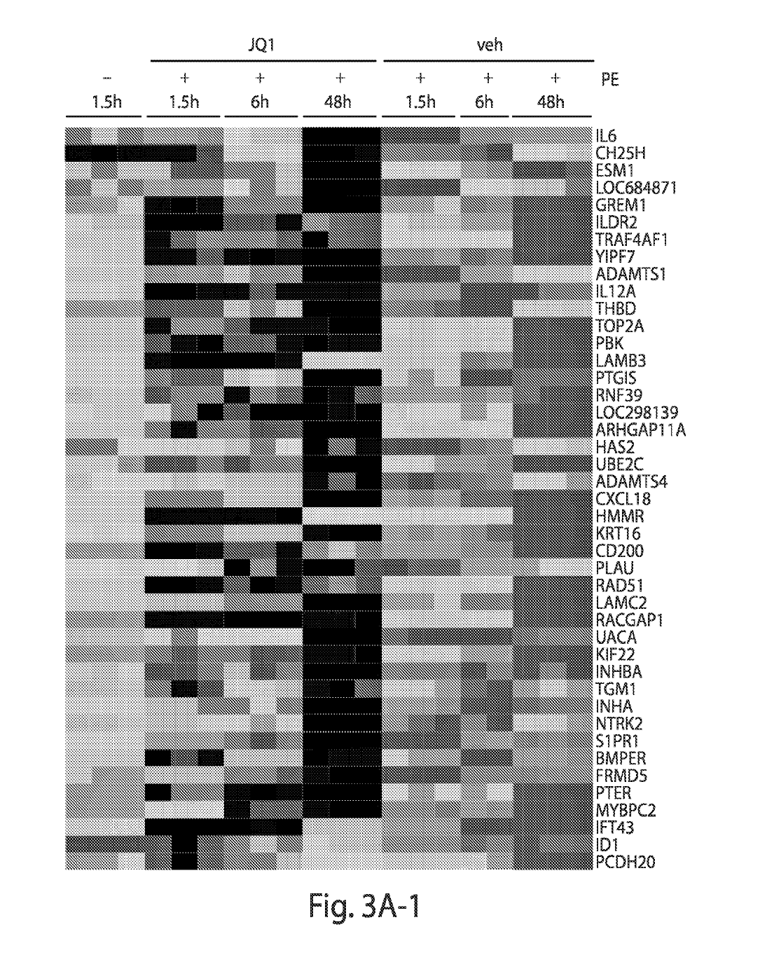

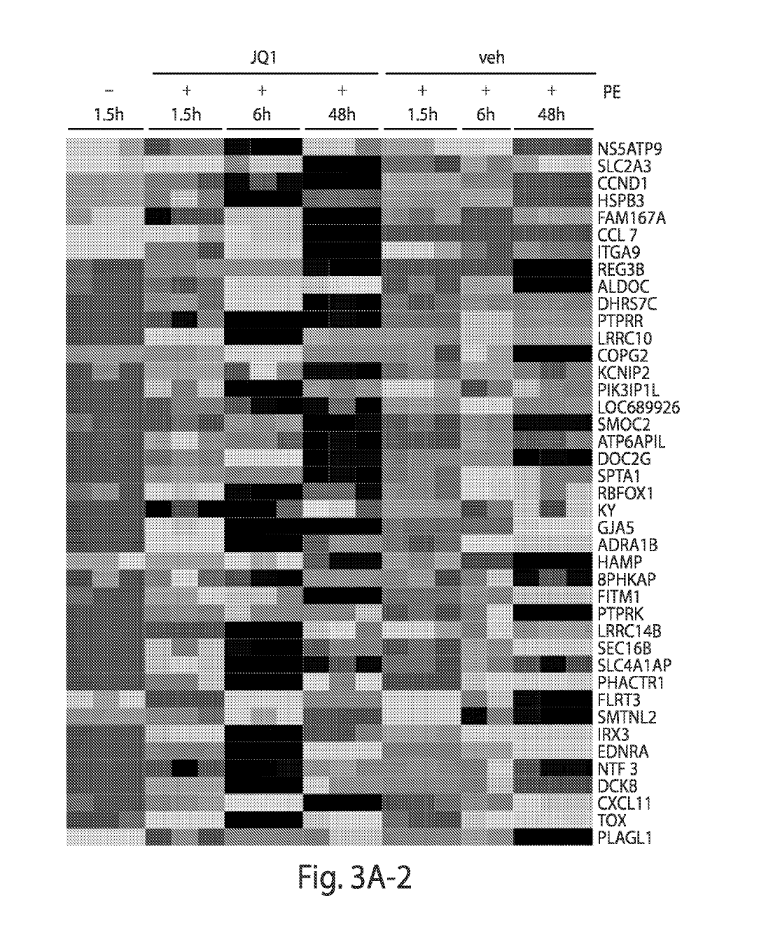

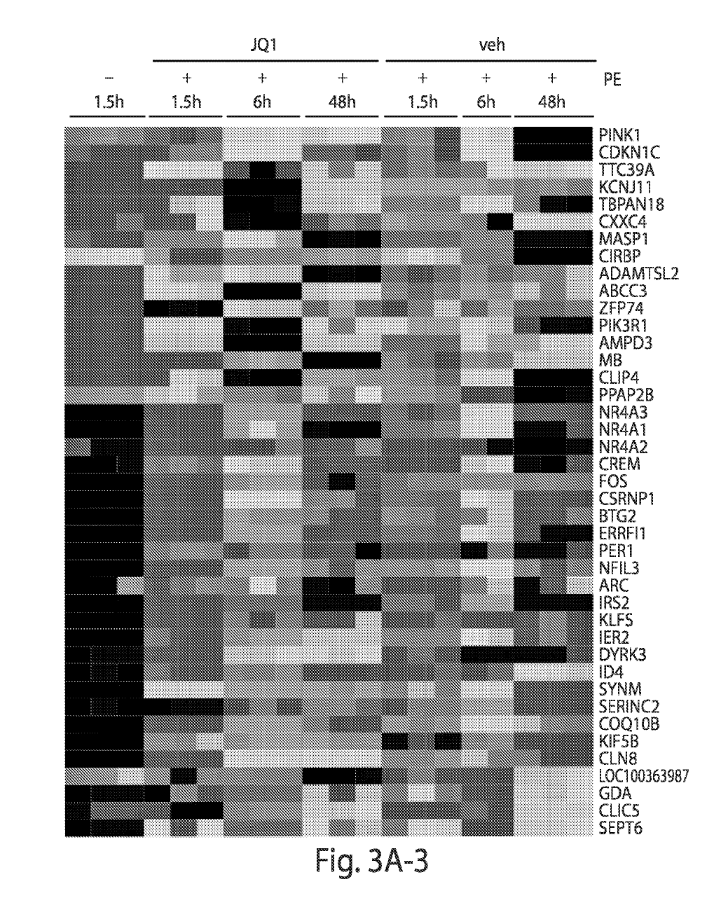

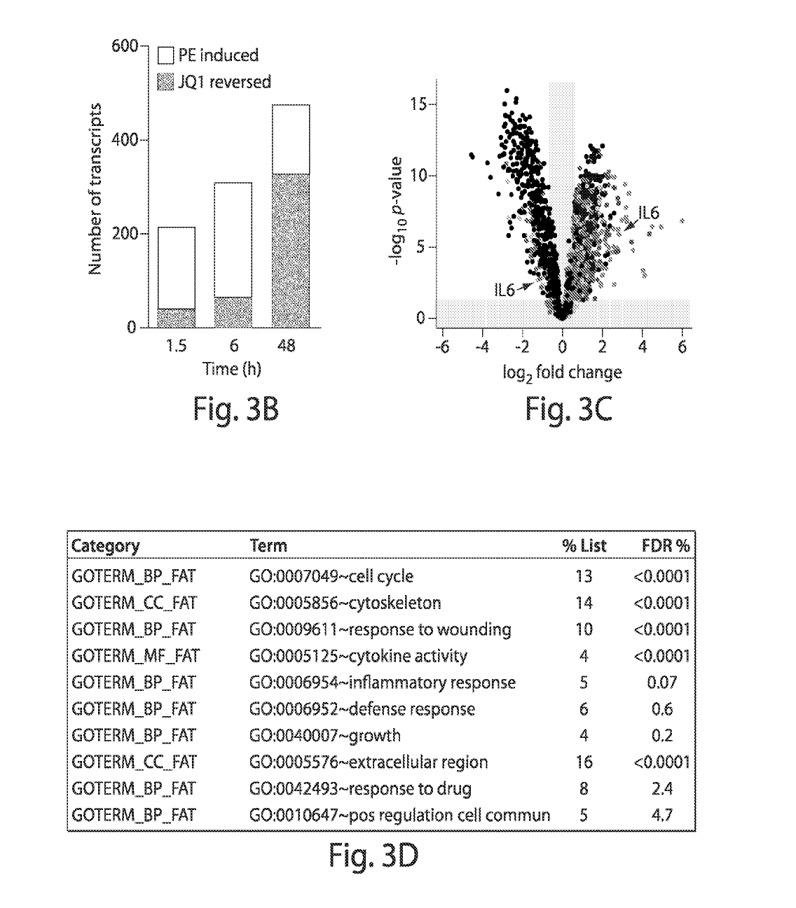

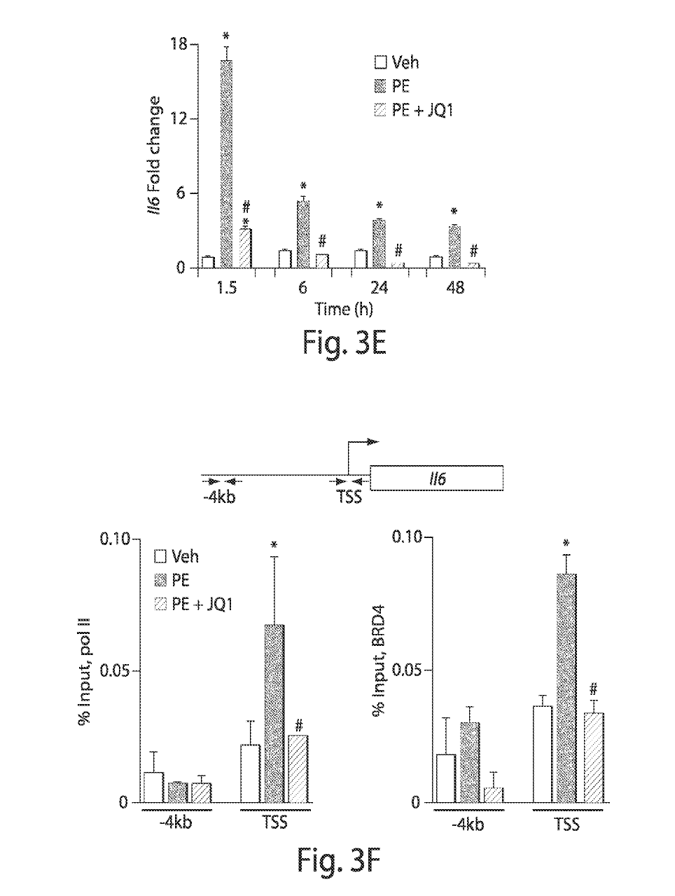

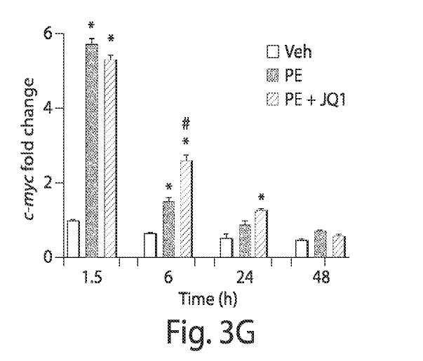

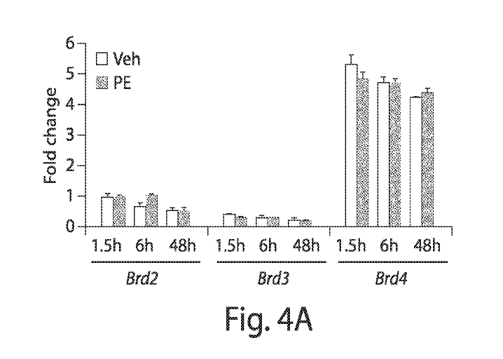

To determine the transcriptional effects of BET bromodomain inhibition during hypertrophic transformation, gene expression profiling (GEP) studies were performed in cultured NRVM at baseline and after PE stimulation (1.5, 6, 48 h) in the presence or absence of JQ1. These three timepoints were assessed to capture induction of early response genes such as c-Myc (Starksen et al., 1986) and the final hypertrophic gene program. Assessment of differentially expressed transcripts revealed three major clusters: genes that were PE inducible and suppressed by JQ1, genes that were PE inducible and unaffected by JQ1, and genes that were PE suppressed and unaffected by JQ1. A heat map of genes selected based on the highest magnitude of PE-mediated changes illustrates each of these clusters (FIG. 3A). Global analysis of these GEPs revealed that PE stimulation resulted in the cumulative induction of over 450 genes and that the dominant effect of JQ1 was to attenuate or completely abrogate PE-mediated gene induction. These transcriptional effects were evident at 1.5 hours and increased over time (FIG. 2B-C), findings consistent with the known role of BETs as essential co-activators of inducible gene expression programs. Functional pathway analysis of the PE-inducible transcripts that were suppressed by JQ1 revealed that BETs play an essential role in a host of biological processes known to be involved in pathologic cardiomyocyte activation, including cytoskeletal reorganization, extracellular matrix production, cell-cycle reentry, paracrine/autocrine stimulation of cellular growth, and pro-inflammatory signaling (FIG. 3D) (Song et al., 2012; Zhao et al., 2004). Using the pro-hypertrophic cytokine IL6 as a representative target (del Vescovo et al., 2013), we confirmed by qRT-PCR that JQ1 significantly attenuated its PE-mediated induction (FIG. 3E). Increased activity of BETs during pathologic stress was not due to PE-mediated increases in their own expression (FIG. 4A). Chromatin immunoprecipitation (ChIP) studies demonstrated that endogenous BRD4 and Pol II were recruited to the proximal promoter of IL6 in response to PE, while JQ1 blocked this recruitment (FIG. 3F). Interestingly, BET bromodomain inhibition did not affect PE-mediated induction of c-Myc (FIG. 3G), an important target regulated directly by BETs in certain myeloid tumors (Delmore et al., 2011; Ott et al., 2012; Toyoshima et al., 2012; Zuber et al., 2011) that is also an established transcriptional driver of pathologic cardiac hypertrophy (Starksen et al., 1986; Xiao et al., 2001; Zhong et al., 2006). Collectively, these in vitro data (FIGS. 2 and 3) demonstrate that BET bromodomain containing proteins regulate cardiomyocyte hypertrophy in a cell-autonomous manner via co-activation of a broad, but specific transcriptional program.

### BET Bromodomain Inhibition Arrests Pathologic Hypertrophy and Heart Failure In Vivo.

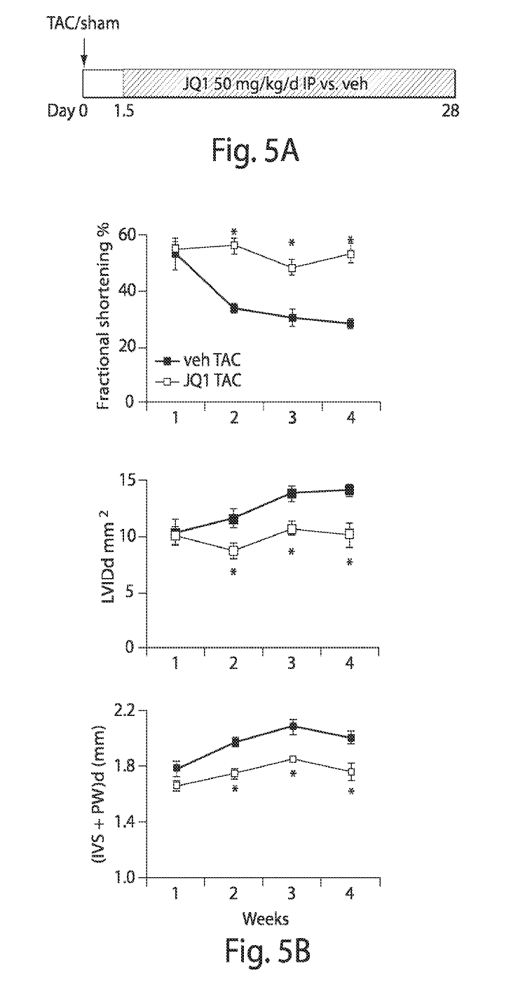

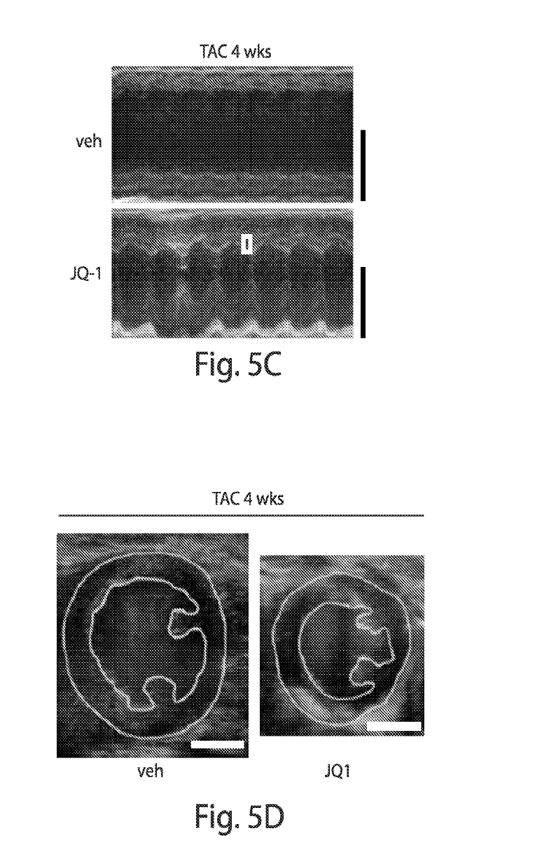

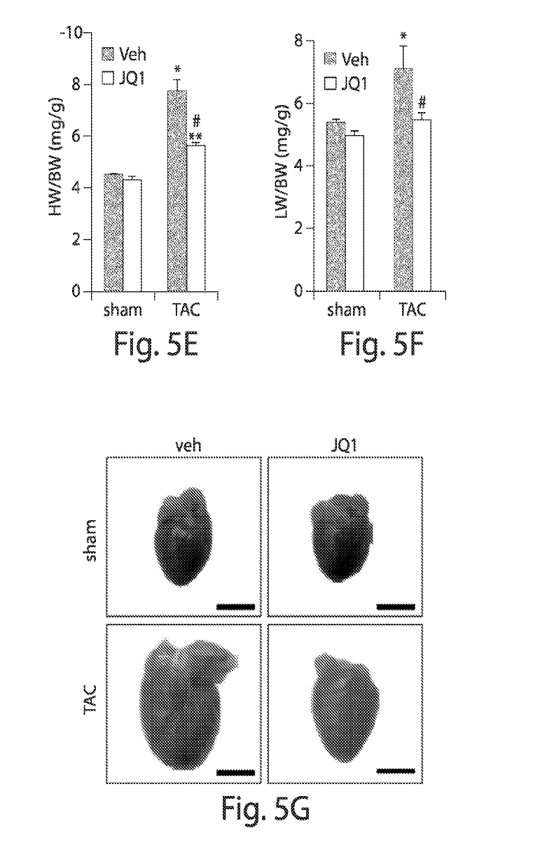

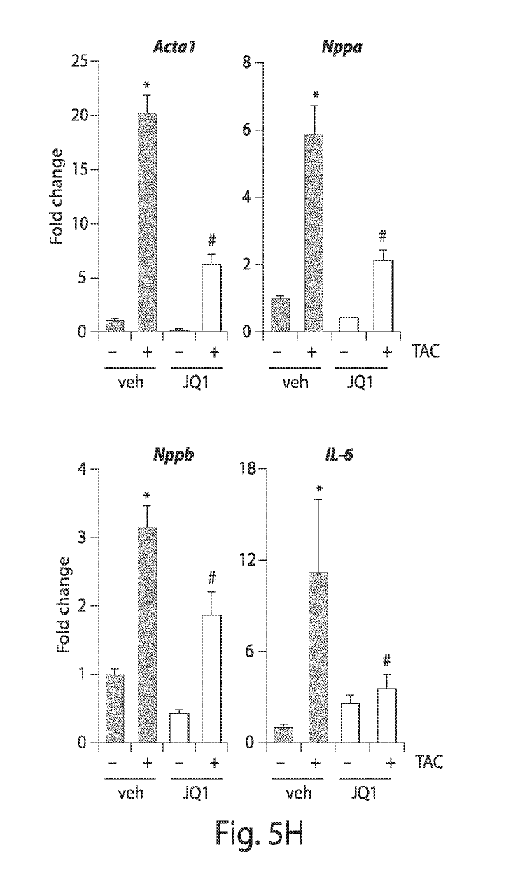

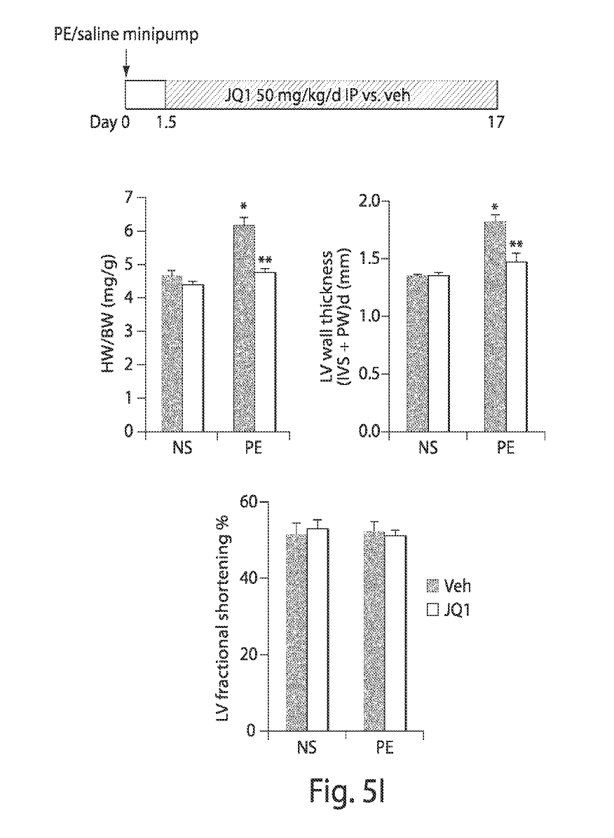

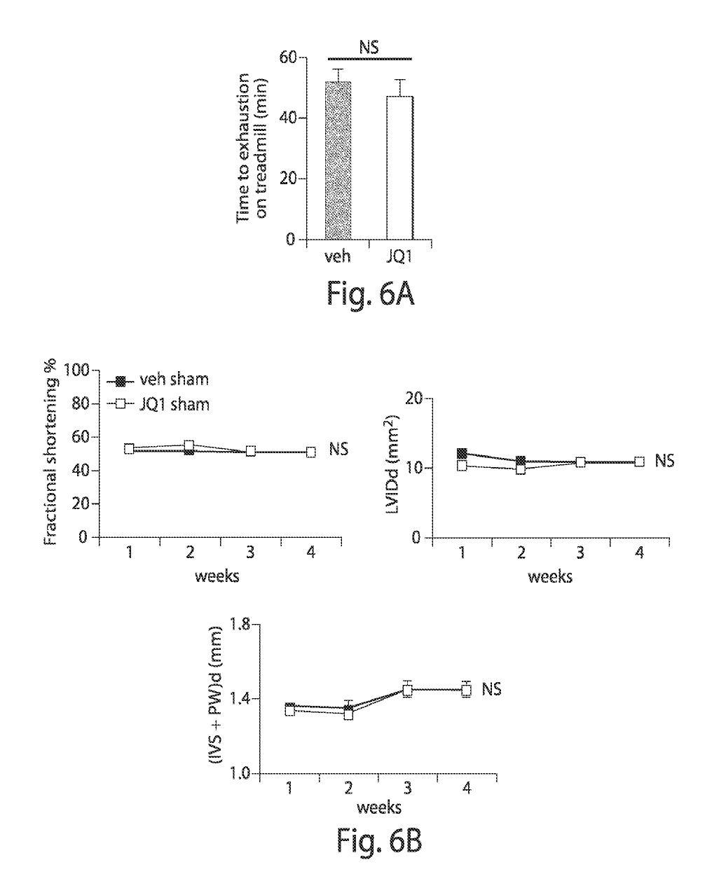

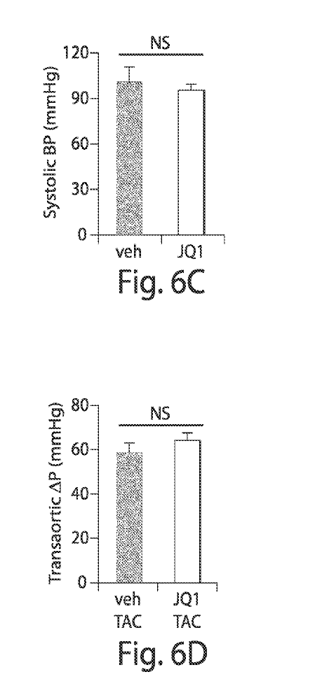

Given our observations in cultured cardiomyocytes, it was hypothesized that BETs might regulate pathologic cardiac remodeling in the intact organism. The favorable therapeutic index of JQ1 was leveraged, which has previously been shown to potently inhibit BET bromodomain function in adult mice without significant toxicity when administered chronically at 50 mg/kg/day (Delmore et al., 2011; Filippakopoulos et al., 2010; Matzuk et al., 2012). In an independent assay, this lack of major toxicity was confirmed by demonstrating that mice treated with this dose of JQ1 for 2-3 weeks had preserved endurance exercise capacity (FIG. 6A), a metric of global cardiometabolic health. For in vivo studies, transverse aortic constriction (TAC) was used, a well characterized model that provides focal hemodynamic stress to the heart and recapitulates several cardinal aspects of pathologic hypertrophy and HF in humans (Rockman et al., 1991). Adult mice subject to TAC develop concentric left ventricular hypertrophy (LVH) by 7-10 days and progress to advanced heart failure after 3-4 weeks. TAC or sham surgery was performed followed by administration of JQ1 (50 mg/kg/day versus an equivalent volume of vehicle) approximately 1.5 days after initiation of TAC (FIG. 5A). Serial echocardiography showed that JQ1 protected against TAC-mediated LV systolic dysfunction, cavity dilation, and wall thickening with effects that were sustained out to 4 weeks (FIG. 5B-D, FIG. 6B). JQ1 treatment also inhibited pathologic cardiomegaly (FIG. 5E; representative photos shown in FIG. 5G), pulmonary congestion (FIG. 5F), and myocardial expression of canonical hypertrophic marker genes (FIG. 5H) after TAC. JQ1 was well tolerated during TAC, as evidenced by normal activity, and lack of significant mortality or weight loss when compared to vehicle treated mice (data not shown). In addition, JQ1 had no adverse effect on LV structure or function in sham treated mice (FIGS. 5E-G and FIG. 6A). Importantly, JQ1 does not affect systemic blood pressure (FIG. 6C). Furthermore, the protective effects of JQ1 in the TAC model were not associated with differences in the pressure gradient across the aortic constriction (FIG. 6D).

In addition to hemodynamic stress, excessive neurohormonal activation is also a central driver of pathologic cardiac hypertrophy (Hill and Olson, 2008; van Berlo et al., 2013). Therefore, it was assessed whether JQ1 could ameliorate pathology in a mouse model of neurohormonally-mediated cardiac hypertrophy. Mice were implanted with osmotic minipumps delivering phenylephrine (PE, 75 mg/kg/day vs. normal saline) followed by JQ1 or vehicle administration begun 1.5 days after minipump installation. This infusion protocol typically produces robust concentric LVH in 2-3 weeks, but does not cause significant LV cavity dilation or depression of LV systolic function in wild type mice. Concordant with the TAC results above, JQ1 potently suppressed the development of pathologic cardiac hypertrophy during chronic PE infusion, without any compromise in LV systolic function (FIG. 5I).

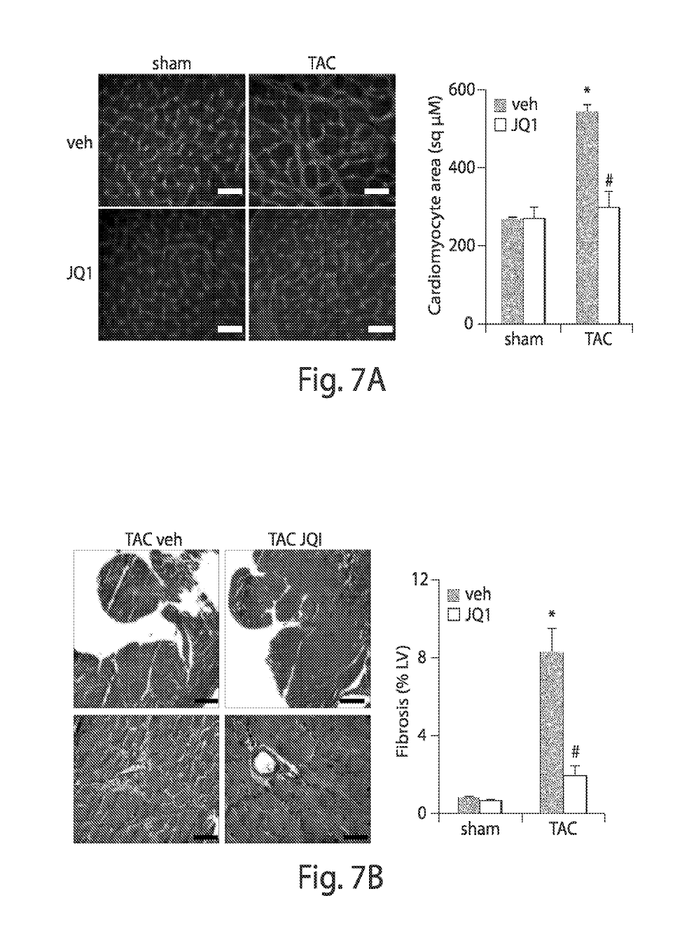

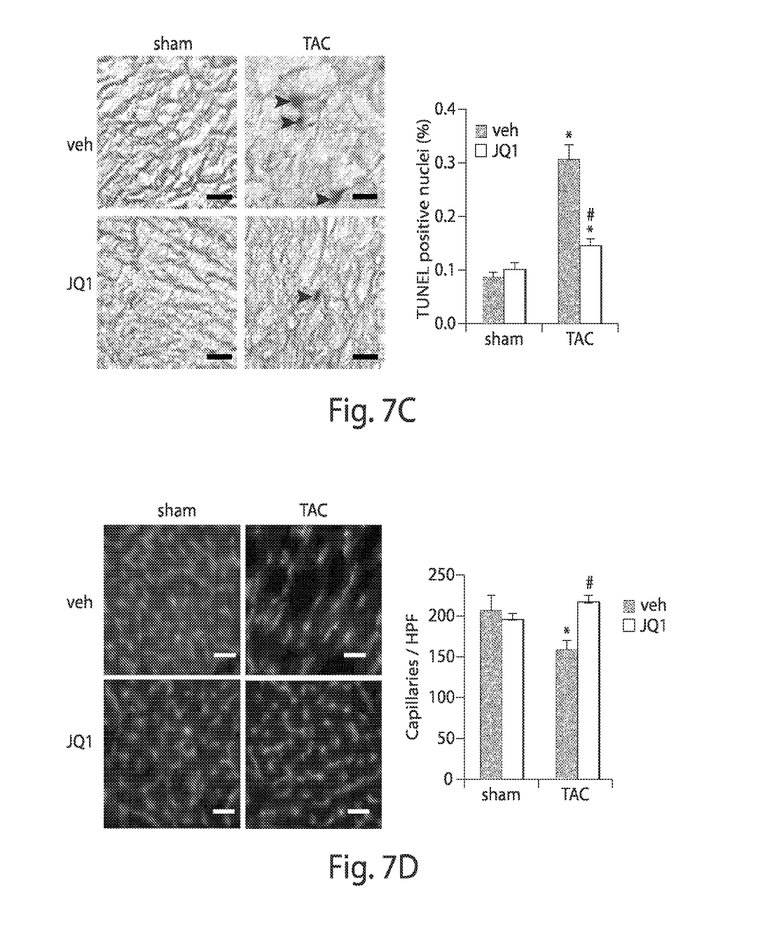

In addition to its favorable effects on cardiac function, it was assessed whether JQ1 also ameliorated cardinal histopathologic features of HF in vivo. Analysis of heart tissue demonstrated that JQ1 significantly attenuated the development of cardiomyocyte hypertrophy (FIG. 7A), myocardial fibrosis (FIG. 7B), apoptotic cell death (FIG. 7C), and capillary rarefaction (FIG. 7D) typically seen after 4 weeks of TAC (Sano et al., 2007; Song et al., 2010). Taken together, the results in FIGS. 5 and 7 show that BET function is critical for the development of pathologic cardiac remodeling in vivo under both hemodynamically and neurohormonally mediated stress. Further, these data establish that selective BET bromodomain inhibition with the small molecule JQ1 is well tolerated and efficacious in animal models of heart failure.

### BET Inhibition Suppresses a Pathologic Cardiac Gene Expression Program In Vivo.

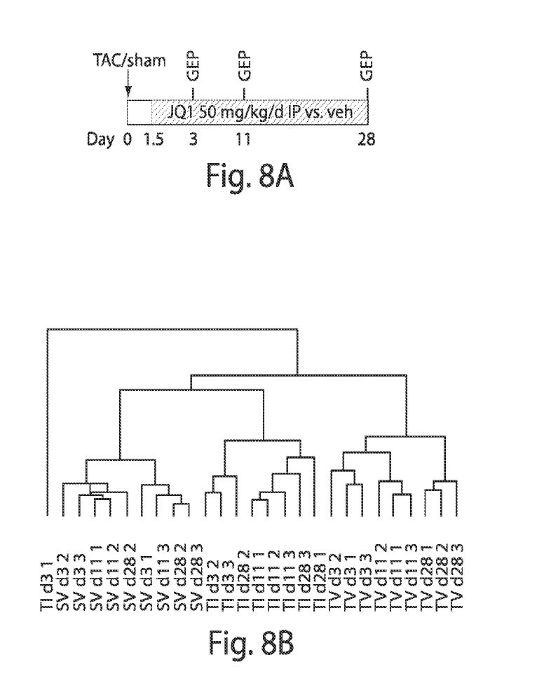

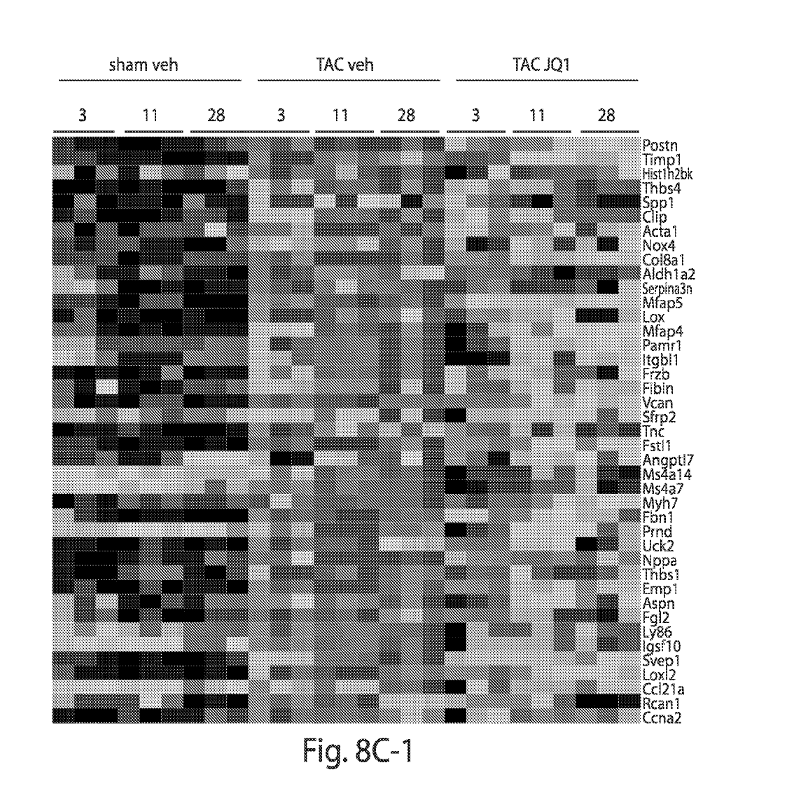

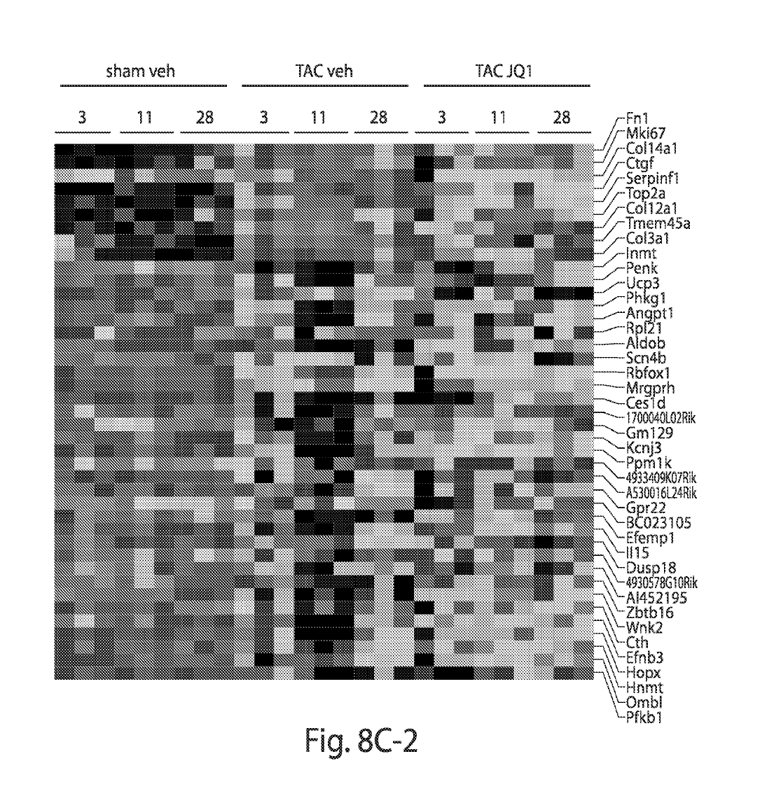

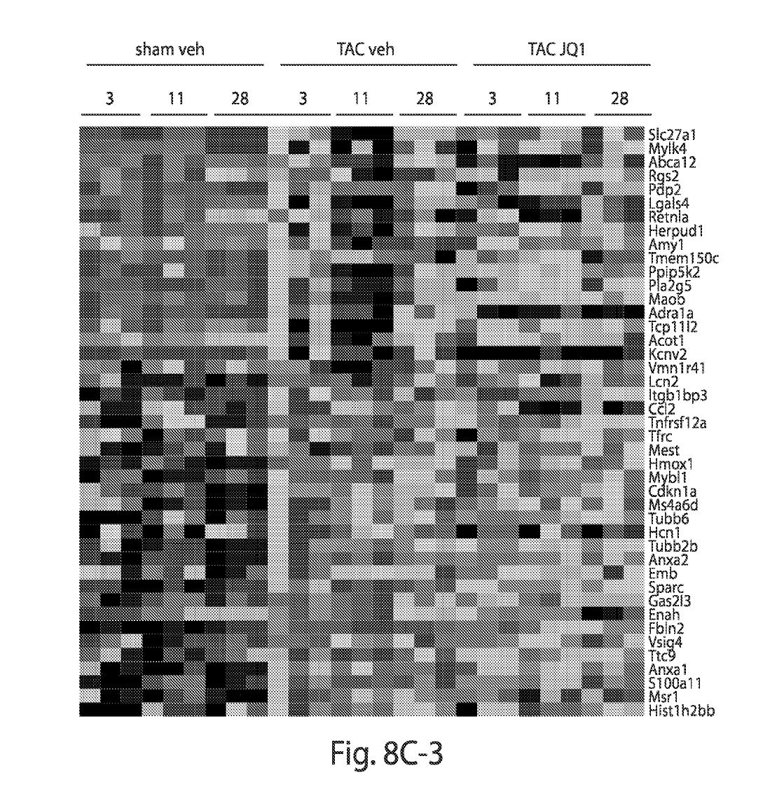

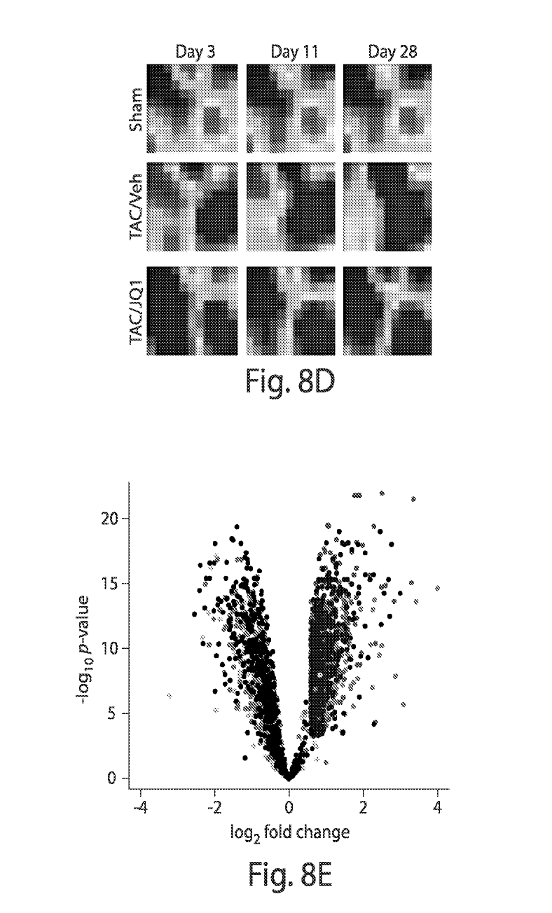

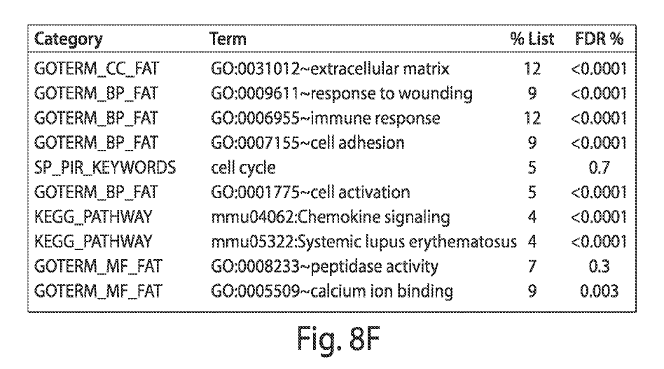

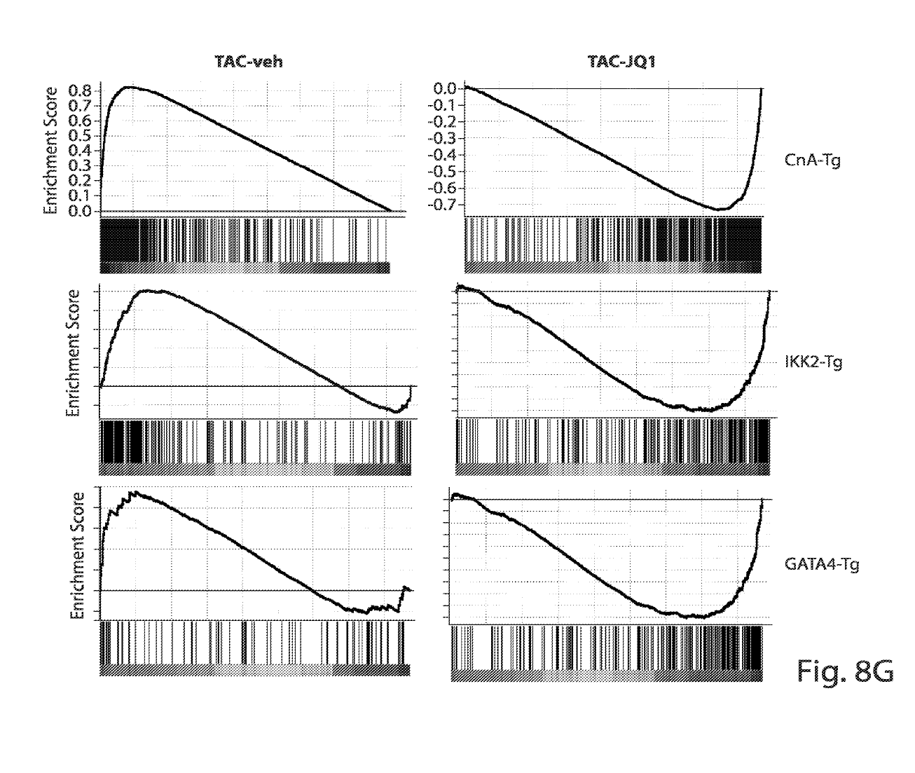

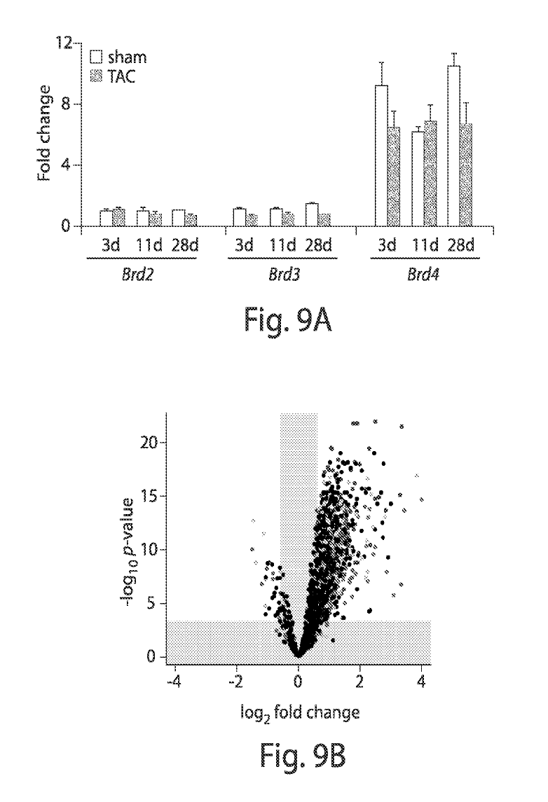

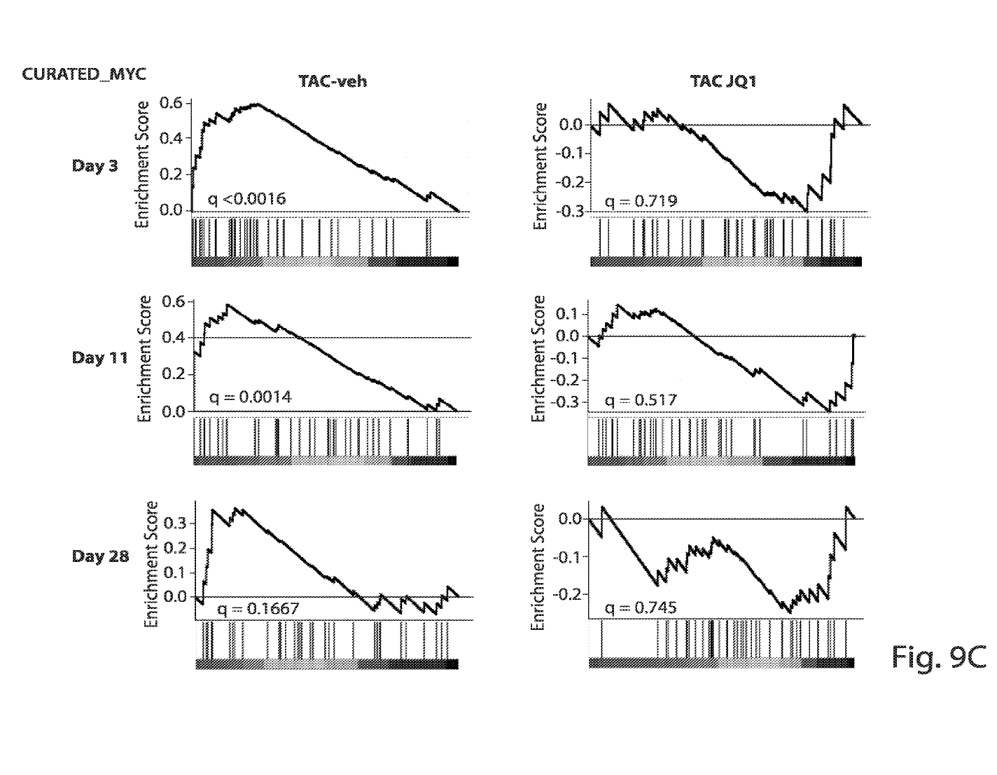

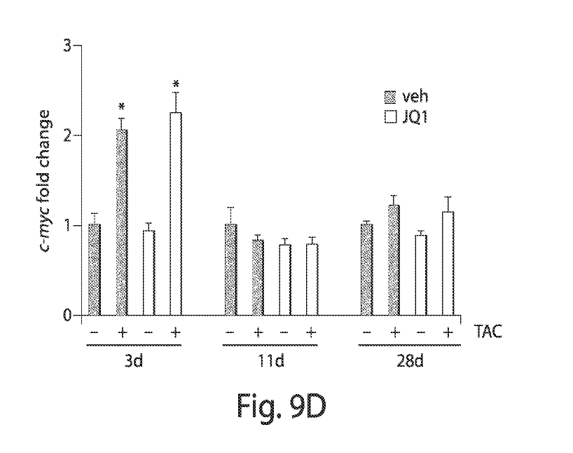

To better understand the mechanism by which BETs regulate stress-induced pathologic remodeling in vivo, kinetic GEP of mouse myocardial tissue was performed. Using the TAC model, microarrays in 3 groups was performed (sham-vehicle, TAC-vehicle, and TAC-JQ1) at 3 timepoints (FIG. 2B): 3 days (to reflect early events that occur prior to the onset of hypertrophy), 11 days (established hypertrophy), and 28 days (advanced pathologic remodeling with signs of HF). Unsupervised hierarchical clustering of GEPs revealed that the TAC-vehicle group had a distinct transcriptomic signature that evolved with time when compared to the sham-vehicle group (FIG. 2B). In contrast, the TAC-JQ1 GEP clustered with the sham group and displayed no significant temporal change despite continuous exposure to TAC (FIG. 2B). Hence, JQ1 treatment suppressed the evolution of a broad pathological gene expression program in the heart, with effects evident as early as 3 days post-TAC. Similar to our GEP in isolated cardiomyocytes (FIG. 3), global analysis of differentially expressed transcripts revealed three major clusters: genes that were TAC-inducible and suppressed by JQ1, those that were TAC inducible and unaffected by JQ1, and those that were TAC suppressed and unaffected by JQ1. A representative heat map of genes (selected for the highest magnitude of TAC-mediated change) highlights each of these three clusters (FIG. 8C). TAC did not significantly alter myocardial expression of Brd2, Brd3, or Brd4 themselves (FIG. 9A). To visualize the global transcriptomic effects of TAC and BET bromodomain inhibition in the model over time, Gene Expression Dynamics Inspector (GEDI) analysis (Eichler et al., 2003) was performed. While the sham mosaic remained temporally invariant, TAC resulted in a progressive induction of clusters of genes over time, indicated by increased signal in numerous tiles within the mosaic (FIG. 8D). BET bromodomain inhibition suppressed the temporal evolution of this TAC-induced, pathologic transcriptional program with a mosaic signature that more closely resembled the sham group (FIG. 8D). Functional pathway analysis of TAC-inducible transcripts that were suppressed by JQ1 showed enrichment for key biological processes involved in pathologic myocardial remodeling and heart failure progression in vivo, including extracellular matrix elaboration, cell cycle reentry, pro-inflammatory activation, and chemokine/cytokine signaling (FIG. 8G) (Song et al., 2012; Zhao et al., 2004). Importantly, these functional terms aligned with the data from isolated NRVM (FIG. 3D) and represent pathologic processes universally observed in advanced human HF (Hannenhalli et al., 2006; Lin et al., 2011).

Stimulus-coupled gene induction occurs via a dynamic interplay between DNA-binding transcription factors and changes in higher-order chromatin structure (Lee and Young, 2013; Schreiber and Bernstein, 2002). Given the broad effects on myocardial gene expression seen with JQ1, it was hypothesized that BETs enable pathologic gene induction via their ability to coordinately co-activate multiple transcription factor pathways in vivo. Using gene set enrichment analysis (GSEA) (Subramanian et al., 2005), our set of TAC-inducible genes that were suppressed by BET inhibition, were compared against compendia of transcription factor signatures. Specifically, GSEA was performed against: (a) The Broad Institute Molecular Signatures Database C3 motif gene sets (Xie et al., 2005) as well as (b) three independent GEPs driven by cardiomyocyte-specific activation of nodal pro-hypertrophic transcriptional effectors in vivo—Calcineurin-NFAT (Bousette et al., 2010), NFκB (Maier et al., 2012) and GATA4 (Heineke et al., 2007). These analyses revealed that the TAC induced gene expression profile was positively enriched for IRF and Ets motifs (q<0.0001) as well as myocardial signatures that result from Calcineurin, NFκB, and GATA4 activation (FIG. 8G). Conversely, the effect of JQ1 demonstrated strong negative enrichment for these same TF signatures (FIG. 5G). In contrast, while TAC was strongly correlated with both c-Myc and E2F signatures, there was no correlation between c-Myc/E2F and JQ1 effect at any timepoint (FIG. 9B; data not shown for E2F). Consistent with our NRVM studies, it was also found that JQ1 had no effect on TAC-mediated induction of c-Myc expression in vivo (FIG. 9C). Hence, these GSEA support a model in which BET bromodomains facilitate gene induction via co-activation of broad, but specific myocardial transcription factor networks.

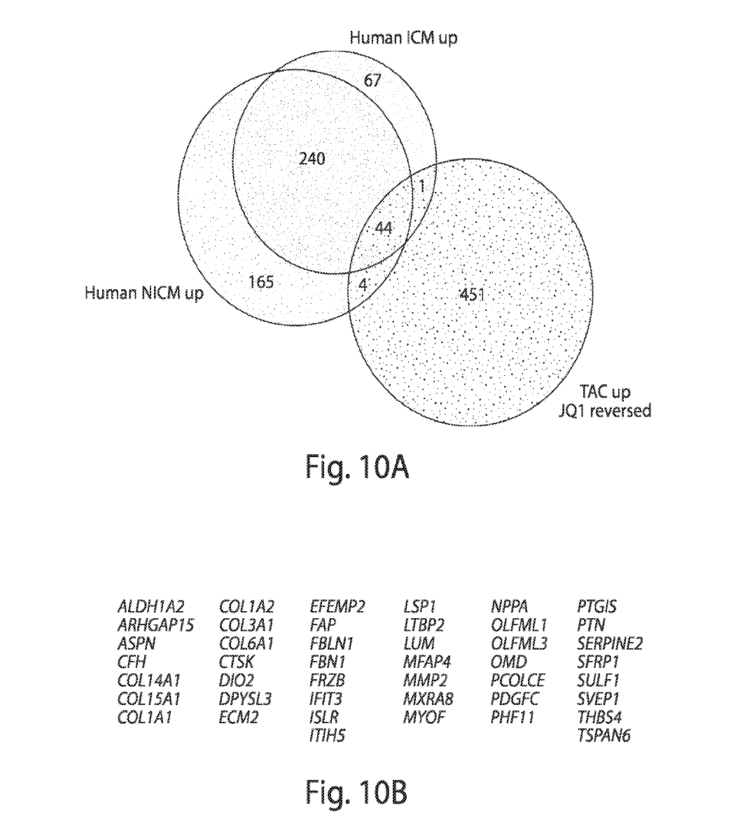

Next the set of TAC-inducible genes that were suppressed by JQ1 were compared against validated gene expression profiles of advanced non-ischemic and ischemic heart failure in humans (Hannenhalli et al., 2006). This analysis demonstrated that targets of BETs in the mouse TAC model overlapped in a statistically significant manner with the set of genes induced in human heart failure (FIG. 10A; χ²<2×10⁻¹⁴). Interestingly, the vast majority (90%) of these targets were common to both ischemic and non-ischemic human heart failure (FIG. 10B). Thus, inasmuch as the gene expression profiles of mice subjected to TAC overlap with that of advanced heart failure in a human cohort, it was found that the transcriptional targets of BET-signaling in mice were also relevant in human disease.

### JQI Ameliorates Pre-Established Pathologic Remodeling in Mouse TAC Model

Adult mice were subjected to pressure overload using transverse aortic constriction (TAC). JQ1 or vehicle was begun on day 18 post-TAC, a time point when significant pathology has already developed (FIG. 11). JQ1 significantly attenuates the progression of (FIG. 11B) LV systolic dysfunction, (FIG. 11C) LV cavity dilation, (FIG. 11D) LV wall thickening, and (FIG. 11E) cardiomegaly, even when administered after significant cardiac pathology has already developed. (N=6-12 per group). This data substantiates the efficacy of BET bromodomain inhibition in an experimental setting that is highly relevant to pre-established cardiac disease in humans. For example, patients typically present with pre-existing or established cardiac hypertrophy and/or heart failure. This data shows that BET bromodomains inhibition with JQ1 is effective even in the setting of pre-established cardiac hypertrophy and heart failure.

JQ1 Inhibits Pathologic Remodeling after Large Anterior MI in Mice (n=5 and n=5-10)

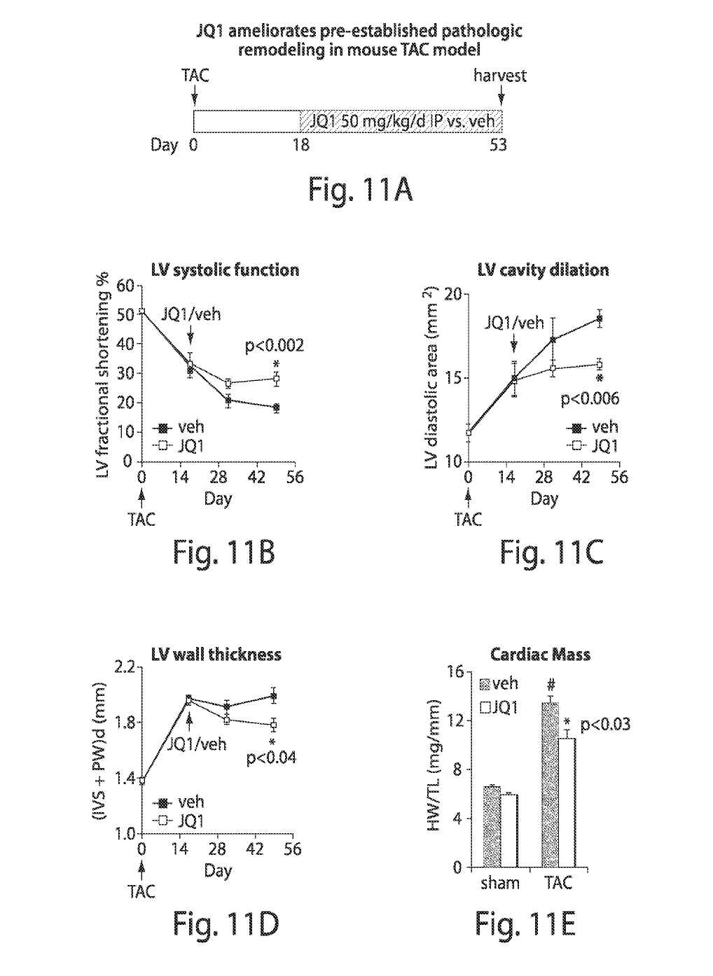

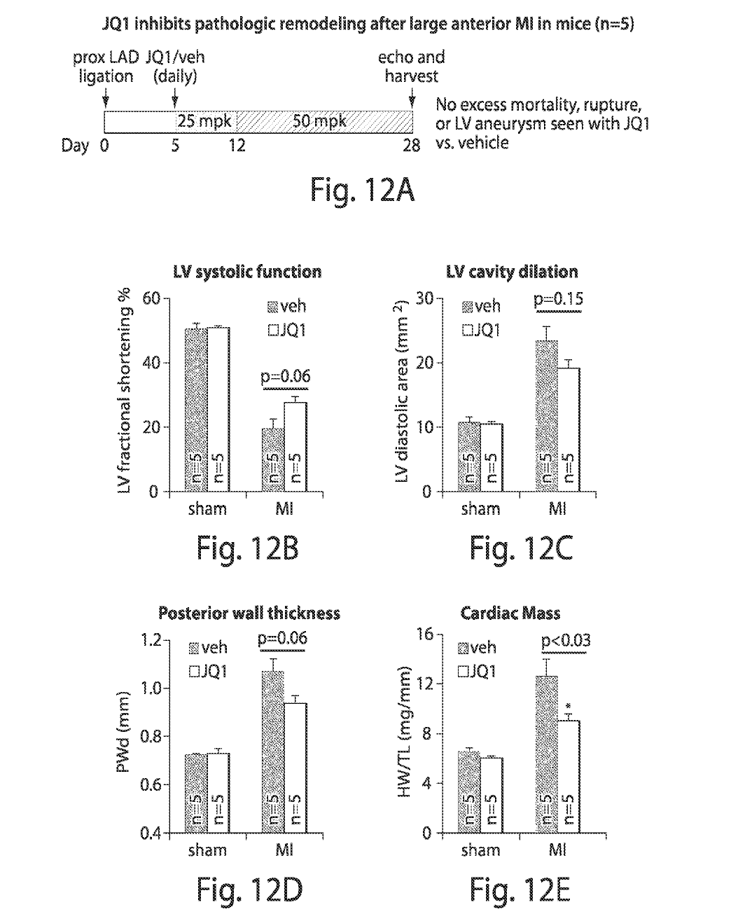

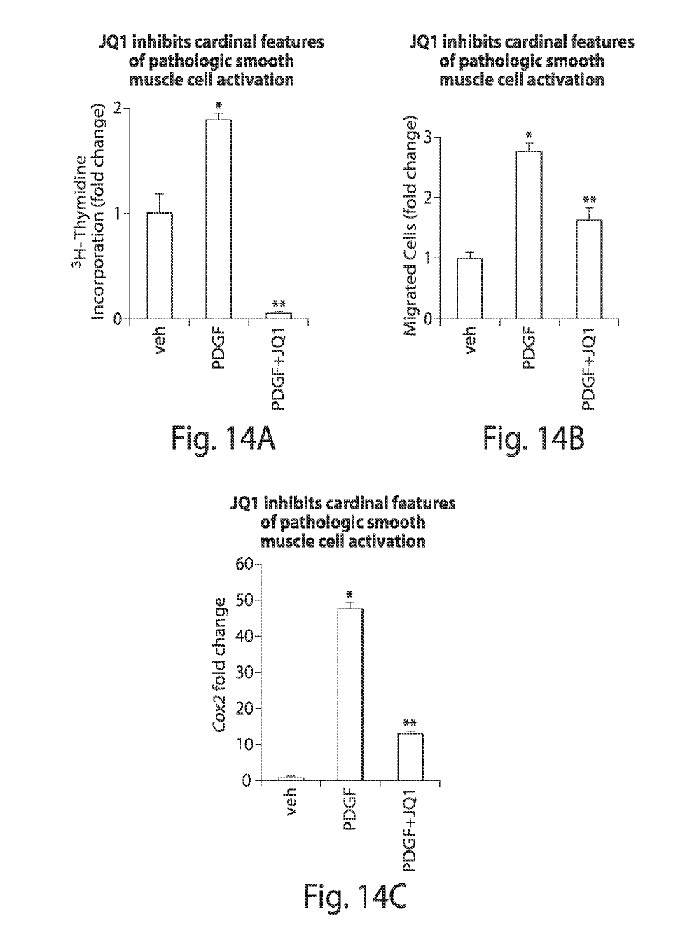

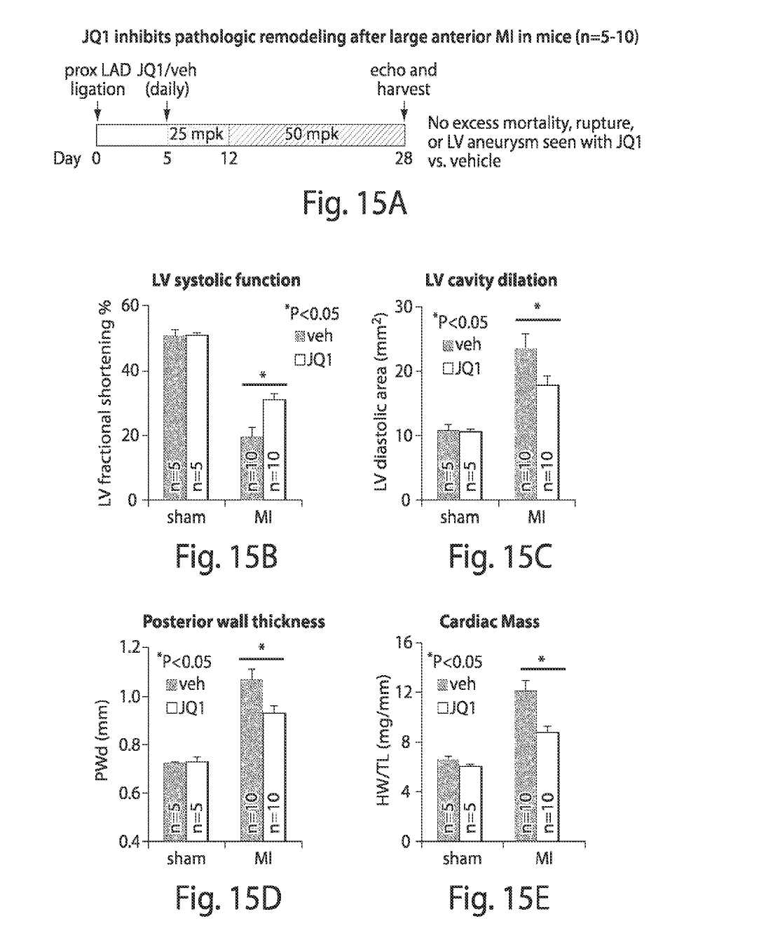

Mice were subjected to permanent proximal LAD ligation to create a large anterior wall myocardial infarction (MI). JQ1 or vehicle was begun at the indicated doses (25 mg/kg/day or 50 mg/kg/day, intraperitoneal injection) on postoperative day 5. No excess mortality, myocardial rupture, and LV aneurysm formation was seen with JQ1 vs. vehicle control with this dosing regimen (FIG. 12). JQ1 attenuates the development of (FIGS. 12B and 15B) LV systolic dysfunction, (FIGS. 12C and 15C) LV cavity dilation, (FIGS. 12D and 15D) LV wall thickening, and (FIGS. 12E and 15E) cardiomegaly after a large anterior wall myocardial infarction. (FIG. 12—N=5 per group; FIG. 15—N=5 in sham group, N=10 in MI group). This data substantiates the efficacy of BET bromodomain inhibition in an experimental setting that is highly relevant to human disease. After a myocardial infarction, abnormal remodeling of the heart occurs in distant areas of non-infarcted myocardium, leading to cardiac dilation, enlargement, and contractile dysfunction. This is a very common cause of heart failure. This data shows that BET bromodomains inhibition protects the non-infarcted regions of myocardium from pathologic remodeling, and therefore preserves overall cardiac function. Neither this model nor the TAC models involve atherosclerosis. Therefore, the ability to protect the heart in these settings are unrelated to any effects on atherosclerosis. These data demonstrate efficacy of BET bromodomain inhibition (using JQ1) in pathologic cardiac remodeling in a mouse model of myocardial infarction (MI). Statistically significant effects were achieved in several major parameters of pathologic post-MI remodeling.

JQ1 Inhibits Doxorubicin Mediated Apoptosis in Cultured Cardiomyocytes.

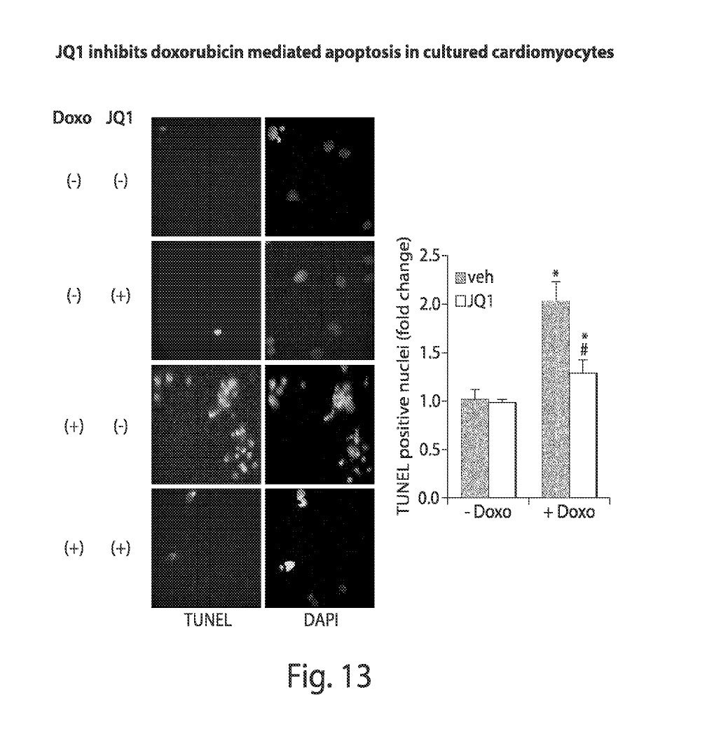

Doxorubicin (Doxo) is an anthracycline compound commonly used as cytotoxic chemotherapy for cancer. Doxo causes dose-dependent toxicity to cardiomyocytes and can cause cardiac enlargement, fibrosis and heart failure in patients. Cardiotoxicity is dose-limiting for anthracyclines such as Doxorubicin and Daunorubicin. FIG. 13 demonstrates that BET bromodomains inhibition with JQ1 blocks Doxo induced cardiotoxicity in cultured cardiomyocytes. Neonatal rat ventricular cardiomyocytes (NRVM) were treated with or without JQ1 (250 nM) for 3 hours, followed by treatment±Doxo (1 μM) for another 24 hours. Cells were assayed for apoptosis by TUNEL staining and nuclei were counterstained with DAPI. Images were taken on a fluorescent microscope and TUNEL positive nuclei were quantified (n=5; * p<0.05 vs. vehicle, (−) Doxo; #p<0.05 vs. vehicle, (+) Doxo. These data support that BET bromodomain inhibition with JQ1 can protect the heart from cardiotoxic chemicals such as anthracyclines. These data support the utility of JQ1 as a cardioprotective agent during cancer therapy with the added benefit that JQ1 also has anti-cancer properties.

### JQ1 Inhibits Cardinal Features of Pathologic Smooth Muscle Cell Activation

All experiments were performed with primary Rat Aortic Smooth Muscle Cells (RASMC), PDGF-bb (10 ng/mL), and JQ1 (500 nM). JQ1 blocks hallmark features of pathologic smooth muscle activation in response to the agonist PDGF-bb such as (FIG. 14A) proliferation (quantified by radiolabeled thymidine incorporation), (FIG. 14B) migration (quantified using a Transwell migration assay), and (FIG. 14C) pathologic gene induction (qRT-PCR shown for Ptgs2/Cox2). These findings support the efficacy of BET bromodomain inhibition against pathologic smooth muscle growth (n=3-6 per group; *p<0.05 vs. vehicle; **p<0.05 vs. PDGF-bb).

### DISCUSSION

The current work implicates BET bromodomain reader proteins as essential regulators of pathological cardiac remodeling and heart failure progression. Studies in cultured cardiomyocytes using Brd4 knockdown and small molecule BET inhibitors establish a cell autonomous role for these proteins in cardiomyocyte hypertrophy. It was further demonstrated that JQ1, a small molecule that specifically disrupts the interaction of BET bromodomains with acetylated chromatin, potently attenuates the development of pathologic hypertrophy and HF in two independent mouse models. Gene expression profiling and ChIP studies reveal that BETs regulate a broad program of pathologic targets via their ability to co-activate key pro-hypertrophic transcriptional networks and recruit Pol II to promoters. In contrast to observations in several cancers (Delmore et al., 2011; Ott et al., 2012; Toyoshima et al., 2012; Zuber et al., 2011), BETs do not directly regulate expression or function of c-Myc in the myocardium, thus providing additional evidence that the transcriptional functions of BETs are highly context specific.

Our gene expression profiles in cultured cardiomyocytes and mouse hearts clearly demonstrate that BETs have target-specificity in the myocardium (FIGS. 3 and 8). Given the genome-wide changes in histone acetylation that occur during cell state transitions in development, differentiation and disease (Lee and Young, 2013), the mechanisms that confer specificity to BET-dependent signaling are important unresolved issues in this rapidly evolving field. It is likely that a combination of post-translational modifications of BETs and other protein interactions with BETs serve as additional determinants of their target gene specificity beyond global changes in histone acetylation. Recent work in cancer cells has demonstrated that phosphorylation of BRD4 by casein kinase 2 (CK2) on specific serine residues affects its ability to functionally interact with and co-activate the transcription factor p53 (Wu et al., 2013). Notably, genetic studies in mice demonstrate that CK2 is a positive regulator of cardiac hypertrophy (Eom et al., 2011). It will be important to explore whether stimulus-coupled post-translational modifications such as CK2-mediated phosphorylation of BRD4 also activate BETs in the heart. In addition, the ability of BETs to co-activate certain transcription factor pathways (e.g. NFAT, GATA4, NFκB) but not others (e.g. c-Myc) may derive, in part, from the stimulus-coupled formation of specific protein complexes in the myocardium.

In addition to studies of protein interactions, it will be important to define the chromatin localization of BETs genome wide in the heart under basal versus pathologic conditions (e.g. sham/TAC). Here, it was shown that BETs are recruited to proximal promoters of stress-induced target genes and facilitate Pol II enrichment at transcriptional start sites in cardiomyocytes (FIG. 3F). ChIP-Seq analysis for BETs, Pol II, and key acetyl-histone marks, when compared with the gene expression profiles provided herein, will provide insights into dynamic changes in chromatin state occurring during pathologic stress and how these changes are affected by BET bromodomain inhibition. These chromatin landscapes will reveal whether myocardial BETs such as BRD4 populate both promoter and enhancer regions of stress inducible genes. Furthermore, the effect of JQ1 on Pol II enrichment patterns across the cardiac genome will provide insights into the potential role for BETs in processes such as de novo Pol II recruitment, pause-release of Pol II at poised loci, and transcriptional elongation (Lee and Young, 2013). Finally, bioinformatic analysis of BET chromatin occupancy throughout the genome in conjunction with transcription factor binding will greatly improve our understanding of the interplay between these epigenetic readers and the specific subsets of DNA-binding factors that they co-activate. In light of recent studies that demonstrate prominent enrichment of BRD4 at a subset of cell-specific, master-regulatory enhancers termed “super-enhancers”, it is possible that preferential loading of BETs on putative myocardial super-enhancers also drives selective induction of stress-induced transcriptional programs during heart failure. As feasibility of constructing genome wide chromatin state maps in adult mouse heart tissue is just emerging (Sayed et al., 2013), application of this technique to the field of myocardial BET signaling will be an exciting area of future investigation.

The initiation and progression of heart failure is known to occur via pathological crosstalk between cardiomyocytes, cardiac fibroblasts and other cell types that may populate the stressed myocardium (van Berlo et al., 2013). While the TAC model of HF provides a relatively focal stress to the heart, and JQ1 attenuates pathologic remodeling without effects on blood pressure or hemodynamic load (FIG. 6C-D), we recognize that BET bromodomain inhibition in vivo may be acting not only on cardiomyocytes, but also on cardiac fibroblasts and other cellular constituents of the myocardium. However, our data do establish that while all three BETs are expressed in rodent cardiomyocytes and heart tissue, Brd4 is expressed at the highest levels (FIG. 1A-B). In addition, both BET bromodomain inhibition and Brd4-knockdown in isolated cultured cardiomyocytes attenuate pathologic cardiomyocyte hypertrophy in vitro (FIG. 2). Collectively, these data identify a cell autonomous role for BRD4 in cardiomyocytes and suggest that it is an important target of JQ1 in vivo. Future studies using cell-type and temporally restricted targeting of Brd4 and other BET family members in adult mice will help annotate their gene- and tissue-specific functions in experimental models of heart failure.

Over the last 15 years, gene targeting approaches in mice have indeed provided key insights into the molecular mechanisms governing cardiac hypertrophy and failure (van Berlo et al., 2013). In contemplating such a strategy for the BETs, we note that Brd4-null and Brd2-null zygotes are nonviable and that germline Brd4 haploinsufficiency leads to severe developmental abnormalities (Houzelstein et al., 2002). Therefore, genetic studies of BET loss-of-function in adult mouse models of HF would likely require conditional approaches that are both tissue-specific and inducible. To date, mice harboring conditionally targeted alleles have not been successfully developed for any of the BET genes. Considering the conceptual and technical obstacles encountered with traditional gene targeting, the chemical biological approach used here to probe the function of BET bromodomain proteins in cardiac biology has several advantages. First, this approach allows us to manipulate BET function with temporal precision that is difficult to achieve using current Cre-lox technology. Second, a chemical biological approach transiently disrupts BET bromodomain interactions with chromatin, as compared to permanent loss of gene function using traditional gene deletion methods. Third, unlike strategies that manipulate the enzymatic activity of epigenetic writers (e.g. HATs) or erasers (e.g. HDACs), JQ1 modulates chromatin-based signal transduction without directly affecting post-translational modifications on histones themselves. Fourth, JQ1 inhibits all three BET family members expressed in the heart (BRD2-4) and therefore blocks functional redundancy within this family, a phenomenon that often confounds single-gene targeting approaches. Finally, the chemical biological approach demonstrates that systemic delivery of small molecule probes such as JQ1 is both effective and well-tolerated in experimental heart failure and suggests the utility of pharmacologic BET bromodomain inhibition in this disease

In conclusion, the current work definitively implicates BET epigenetic reader proteins as essential components of the transcriptional machinery that drives pathologic cardiac remodeling and HF. Specifically, we identify BETs as new targets in the myocardium and BET bromodomain inhibition as a promising approach for HF therapy. As JQ1 appears to be well tolerated in mouse models of heart failure, our work provides a rationale for using drug-like BET bromodomain inhibitors as a pharmacologic strategy in this disease. Considering the intense interest in the development of transcriptional therapies in the field (McKinsey and Olson, 2005), this chemical biological approach provides proof-of-principle that modulation of higher-order chromatin state and chromatin-dependent signal transduction can be harnessed for therapeutic gain in heart failure.

### REFERENCES

Arrowsmith, C. H., Bountra, C., Fish, P. V., Lee, K., and Schapira, M. (2012). Epigenetic protein families: a new frontier for drug discovery. Nat Rev Drug Discov 11, 384-400.
Banerjee, C., Archin, N., Michaels, D., Belkina, A. C., Denis, G. V., Bradner, J., Sebastiani, P., Margolis, D. M., and Montano, M. (2012). BET bromodomain inhibition as a novel strategy for reactivation of HIV-1. Journal of leukocyte biology 92, 1147-1154.
Bartholomeeusen, K., Xiang, Y., Fujinaga, K., and Peterlin, B. M. (2012). Bromodomain and extra-terminal (BET) bromodomain inhibition activate transcription via transient release of positive transcription elongation factor b (P-TEFb) from 7SK small nuclear ribonucleoprotein. J Biol Chem 287, 36609-36616.
Bisgrove, D. A., Mahmoudi, T., Henklein, P., and Verdin, E. (2007). Conserved P-TEFb-interacting domain of BRD4 inhibits HIV transcription. Proc Natl Acad Sci USA 104, 13690-13695.
Bousette, N., Chugh, S., Fong, V., Isserlin, R., Kim, K. H., Volchuk, A., Backx, P. H., Liu, P., Kislinger, T., MacLennan, D. H., et al. (2010). Constitutively active calcineurin induces cardiac endoplasmic reticulum stress and protects against apoptosis that is mediated by alpha-crystallin-B. Proc Natl Acad Sci USA 107, 18481-18486.
de Simone, G., Gottdiener, J. S., Chinali, M., and Maurer, M. S. (2008). Left ventricular mass predicts heart failure not related to previous myocardial infarction: the Cardiovascular Health Study. European heart journal 29, 741-747.
del Vescovo, C. D., Cotecchia, S., and Diviani, D. (2013). A-kinase-anchoring protein-Lbc anchors IkappaB kinase beta to support interleukin-6-mediated cardiomyocyte hypertrophy. Mol Cell Biol 33, 14-27.
Delmore, J. E., Issa, G. C., Lemieux, M. E., Rahl, P. B., Shi, J., Jacobs, H. M., Kastritis, E., Gilpatrick, T., Paranal, R. M., Qi, J., et al. (2011). BET bromodomain inhibition as a therapeutic strategy to target c-Myc. Cell 146, 904-917.
Drazner, M. H., Rame, J. E., Marino, E. K., Gottdiener, J. S., Kitzman, D. W., Gardin, J. M., Manolio, T. A., Dries, D. L., and Siscovick, D. S. (2004). Increased left ventricular mass is a risk factor for the development of a depressed left ventricular ejection fraction within five years: the Cardiovascular Health Study. J Am Coll Cardiol 43, 2207-2215.
Eichler, G. S., Huang, S., and Ingber, D. E. (2003). Gene Expression Dynamics Inspector (GEDI): for integrative analysis of expression profiles. Bioinformatics 19, 2321-2322.
Eom, G. H., Cho, Y. K., Ko, J. H., Shin, S., Choe, N., Kim, Y., Joung, H., Kim, H. S., Nam, K. I., Kee, H. J., et al. (2011). Casein kinase-2alpha1 induces hypertrophic response by phosphorylation of histone deacetylase 2 S394 and its activation in the heart. Circulation 123, 2392-2403.
Filippakopoulos, P., Qi, J., Picaud, S., Shen, Y., Smith, W. B., Fedorov, O., Morse, E. M., Keates, T., Hickman, T. T., Felletar, I., et al. (2010). Selective inhibition of BET bromodomains. Nature 468, 1067-1073.
Froese, N., Kattih, B., Breitbart, A., Grund, A., Geffers, R., Molkentin, J. D., Kispert, A., Wollert, K. C., Drexler, H., and Heineke, J. (2011). GATA6 promotes angiogenic function and survival in endothelial cells by suppression of autocrine transforming growth factor beta/activin receptor-like kinase 5 signaling. J Biol Chem 286, 5680-5690.
Gusterson, R. J., Jazrawi, E., Adcock, I. M., and Latchman, D. S. (2003). The transcriptional co-activators CREB-binding protein (CBP) and p300 play a critical role in cardiac hypertrophy that is dependent on their histone acetyltransferase activity. J Biol Chem 278, 6838-6847.
Haldar, S. M., Jeyaraj, D., Anand, P., Zhu, H., Lu, Y., Prosdocimo, D. A., Eapen, B., Kawanami, D., Okutsu, M., Brotto, L., et al. (2012). Kruppel-like factor 15 regulates skeletal muscle lipid flux and exercise adaptation. Proc Natl Acad Sci USA 109, 6739-6744.
Haldar, S. M., Lu, Y., Jeyaraj, D., Kawanami, D., Cui, Y., Eapen, S. J., Hao, C., Li, Y., Doughman, Y. Q., Watanabe, M., et al. (2010). Klf15 deficiency is a molecular link between heart failure and aortic aneurysm formation. Science translational medicine 2, 26ra26.
Hannenhalli, S., Putt, M. E., Gilmore, J. M., Wang, J., Parmacek, M. S., Epstein, J. A., Morrisey, E. E., Margulies, K. B., and Cappola, T. P. (2006). Transcriptional genomics associates FOX transcription factors with human heart failure. Circulation 114, 1269-1276.
Heineke, J., Auger-Messier, M., Xu, J., Oka, T., Sargent, M. A., York, A., Klevitsky, R., Vaikunth, S., Duncan, S. A., Aronow, B. J., et al. (2007). Cardiomyocyte GATA4 functions as a stress-responsive regulator of angiogenesis in the murine heart. J Clin Invest 117, 3198-3210.
Hill, J. A., and Olson, E. N. (2008). Cardiac plasticity. N Engl J Med 358, 1370-1380.
Houzelstein, D., Bullock, S. L., Lynch, D. E., Grigorieva, E. F., Wilson, V. A., and Beddington, R. S. (2002). Growth and early postimplantation defects in mice deficient for the bromodomain-containing protein Brd4. Mol Cell Biol 22, 3794-3802.
Hu, P., Zhang, D., Swenson, L., Chakrabarti, G., Abel, E. D., and Litwin, S. E. (2003). Minimally invasive aortic banding in mice: effects of altered cardiomyocyte insulin signaling during pressure overload. Am J Physiol Heart Circ Physiol 285, H1261-1269.
Hunt, S. A., Abraham, W. T., Chin, M. H., Feldman, A. M., Francis, G. S., Ganiats, T. G., Jessup, M., Konstam, M. A., Mancini, D. M., Michl, K., et al. (2009). 2009 focused update incorporated into the ACC/AHA 2005 Guidelines for the Diagnosis and Management of Heart Failure in Adults: a report of the American College of Cardiology Foundation/American Heart Association Task Force on Practice Guidelines: developed in collaboration with the International Society for Heart and Lung Transplantation. Circulation 119, e391-479.
Lee, T. I., and Young, R. A. (2013). Transcriptional regulation and its misregulation in disease. Cell 152, 1237-1251.
Levy, D., Garrison, R. J., Savage, D. D., Kannel, W. B., and Castelli, W. P. (1990). Prognostic implications of echocardiographically determined left ventricular mass in the Framingham Heart Study. N Engl J Med 322, 1561-1566.
Liang, Q., De Windt, L. J., Witt, S. A., Kimball, T. R., Markham, B. E., and Molkentin, J. D. (2001). The transcription factors GATA4 and GATA6 regulate cardiomyocyte hypertrophy in vitro and in vivo. J Biol Chem 276, 30245-30253.
Lin, D., Hollander, Z., Meredith, A., Stadnick, E., Sasaki, M., Cohen Freue, G., Qasimi, P., Mui, A., Ng, R. T., Balshaw, R., et al. (2011). Molecular signatures of end-stage heart failure. Journal of cardiac failure 17, 867-874.
Liu, Q., Chen, Y., Auger-Messier, M., and Molkentin, J. D. (2012). Interaction between NFkappaB and NFAT coordinates cardiac hypertrophy and pathological remodeling. Circ Res 110, 1077-1086.
Lockwood, W. W., Zejnullahu, K., Bradner, J. E., and Varmus, H. (2012). Sensitivity of human lung adenocarcinoma cell lines to targeted inhibition of BET epigenetic signaling proteins. Proc Natl Acad Sci USA 109, 19408-19413.
Maier, H. J., Schips, T. G., Wietelmann, A., Kruger, M., Brunner, C., Sauter, M., Klingel, K., Bottger, T., Braun, T., and Wirth, T. (2012). Cardiomyocyte-specific IkappaB kinase (IKK)/NF-kappaB activation induces reversible inflammatory cardiomyopathy and heart failure. Proc Natl Acad Sci USA 109, 11794-11799.
Margulies, K. B., Matiwala, S., Cornejo, C., Olsen, H., Craven, W. A., and Bednarik, D. (2005). Mixed messages: transcription patterns in failing and recovering human myocardium. Circ Res 96, 592-599.
Matzuk, M. M., McKeown, M. R., Filippakopoulos, P., Li, Q., Ma, L., Agno, J. E., Lemieux, M. E., Picaud, S., Yu, R. N., Qi, J., et al. (2012). Small-Molecule Inhibition of BRDT for Male Contraception. Cell 150, 673-684.
McKinsey, T. A., and Olson, E. N. (2005). Toward transcriptional therapies for the failing heart: chemical screens to modulate genes. J Clin Invest 115, 538-546.
Molkentin, J. D., Lu, J. R., Antos, C. L., Markham, B., Richardson, J., Robbins, J., Grant, S. R., and Olson, E. N. (1998). A calcineurin-dependent transcriptional pathway for cardiac hypertrophy. Cell 93, 215-228.
Montgomery, R. L., Davis, C. A., Potthoff, M. J., Haberland, M., Fielitz, J., Qi, X., Hill, J. A., Richardson, J. A., and Olson, E. N. (2007). Histone deacetylases 1 and 2 redundantly regulate cardiac morphogenesis, growth, and contractility. Genes Dev 21, 1790-1802.
Ott, C. J., Kopp, N., Bird, L., Paranal, R. M., Qi, J., Bowman, T., Rodig, S. J., Kung, A. L., Bradner, J. E., and Weinstock, D. M. (2012). BET bromodomain inhibition targets both c-MYC and IL7R in high-risk acute lymphoblastic leukemia. Blood.
Passier, R., Zeng, H., Frey, N., Naya, F. J., Nicol, R. L., McKinsey, T. A., Overbeek, P., Richardson, J. A., Grant, S. R., and Olson, E. N. (2000). CaM kinase signaling induces cardiac hypertrophy and activates the MEF2 transcription factor in vivo. J Clin Invest 105, 1395-1406.
Rockman, H. A., Ross, R. S., Harris, A. N., Knowlton, K. U., Steinhelper, M. E., Field, L. J., Ross, J., Jr., and Chien, K. R. (1991). Segregation of atrial-specific and inducible expression of an atrial natriuretic factor transgene in an in vivo murine model of cardiac hypertrophy. Proc Natl Acad Sci USA 88, 8277-8281.
Roger, V. L., Go, A. S., Lloyd-Jones, D. M., Benjamin, E. J., Berry, J. D., Borden, W. B., Bravata, D. M., Dai, S., Ford, E. S., Fox, C. S., et al. (2012). Executive summary: heart disease and stroke statistics—2012 update: a report from the American Heart Association. Circulation 125, 188-197.
Sano, M., Minamino, T., Toko, H., Miyauchi, H., Orimo, M., Qin, Y., Akazawa, H., Tateno, K., Kayama, Y., Harada, M., et al. (2007). p53-induced inhibition of Hif-1 causes cardiac dysfunction during pressure overload. Nature 446, 444-448.
Sayed, D., He, M., Yang, Z., Lin, L., and Abdellatif, M. (2013). Transcriptional regulation patterns revealed by high resolution chromatin immunoprecipitation during cardiac hypertrophy. J Biol Chem 288, 2546-2558.
Schreiber, S. L., and Bernstein, B. E. (2002). Signaling network model of chromatin. Cell 111, 771-778.
Simpson, P., McGrath, A., and Savion, S. (1982). Myocyte hypertrophy in neonatal rat heart cultures and its regulation by serum and by catecholamines. Circ Res 51, 787-801.
Song, H. K., Hong, S. E., Kim, T., and Kim do, H. (2012). Deep RNA sequencing reveals novel cardiac transcriptomic signatures for physiological and pathological hypertrophy. PloS one 7, e35552.
Song, X., Kusakari, Y., Xiao, C. Y., Kinsella, S. D., Rosenberg, M. A., Scherrer-Crosbie, M., Hara, K., Rosenzweig, A., and Matsui, T. (2010). mTOR attenuates the inflammatory response in cardiomyocytes and prevents cardiac dysfunction in pathological hypertrophy. American journal of physiology Cell physiology 299, C1256-1266.
Starksen, N. F., Simpson, P. C., Bishopric, N., Coughlin, S. R., Lee, W. M., Escobedo, J. A., and Williams, L. T. (1986). Cardiac myocyte hypertrophy is associated with c-myc protooncogene expression. Proc Natl Acad Sci USA 83, 8348-8350.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Toyoshima, M., Howie, H. L., Imakura, M., Walsh, R. M., Annis, J. E., Chang, A. N., Frazier, J., Chau, B. N., Loboda, A., Linsley, P. S., et al. (2012). Functional genomics identifies therapeutic targets for MYC-driven cancer. Proc Natl Acad Sci USA 109, 9545-9550.
Trivedi, C. M., Luo, Y., Yin, Z., Zhang, M., Zhu, W., Wang, T., Floss, T., Goettlicher, M., Noppinger, P. R., Wurst, W., et al. (2007). Hdac2 regulates the cardiac hypertrophic response by modulating Gsk3 beta activity. Nat Med 13, 324-331.
van Berlo, J. H., Elrod, J. W., Aronow, B. J., Pu, W. T., and Molkentin, J. D. (2011). Serine 105 phosphorylation of transcription factor GATA4 is necessary for stress-induced cardiac hypertrophy in vivo. Proc Natl Acad Sci USA 108, 12331-12336.
van Berlo, J. H., Maillet, M., and Molkentin, J. D. (2013). Signaling effectors underlying pathologic growth and remodeling of the heart. J Clin Invest 123, 37-45.
Wamstad, J. A., Alexander, J. M., Truty, R. M., Shrikumar, A., Li, F., Eilertson, K. E., Ding, H., Wylie, J. N., Pico, A. R., Capra, J. A., et al. (2012). Dynamic and coordinated epigenetic regulation of developmental transitions in the cardiac lineage. Cell 151, 206-220.
Wang, X., Helfer, C. M., Pancholi, N., Bradner, J. E., and You, J. (2013). Recruitment of brd4 to the human papillomavirus type 16 DNA replication complex is essential for replication of viral DNA. Journal of virology 87, 3871-3884.
Wei, J. Q., Shehadeh, L. A., Mitrani, J. M., Pessanha, M., Slepak, T. I., Webster, K. A., and Bishopric, N. H. (2008). Quantitative control of adaptive cardiac hypertrophy by acetyltransferase p300. Circulation 118, 934-946.
Wu, S. Y., Lee, A. Y., Lai, H. T., Zhang, H., and Chiang, C. M. (2013). Phospho switch triggers brd4 chromatin binding and activator recruitment for gene-specific targeting. Mol Cell 49, 843-857.
Xiao, G., Mao, S., Baumgarten, G., Serrano, J., Jordan, M. C., Roos, K. P., Fishbein, M. C., and MacLellan, W. R. (2001). Inducible activation of c-Myc in adult myocardium in vivo provokes cardiac myocyte hypertrophy and reactivation of DNA synthesis. Circ Res 89, 1122-1129.
Xie, X., Lu, J., Kulbokas, E. J., Golub, T. R., Mootha, V., Lindblad-Toh, K., Lander, E. S., and Kellis, M. (2005). Systematic discovery of regulatory motifs in human promoters and 3′ UTRs by comparison of several mammals. Nature 434, 338-345.
Xu, J., Gong, N. L., Bodi, I., Aronow, B. J., Backx, P. H., and Molkentin, J. D. (2006). Myocyte enhancer factors 2A and 2C induce dilated cardiomyopathy in transgenic mice. J Biol Chem 281, 9152-9162.
Zhang, C. L., McKinsey, T. A., Chang, S., Antos, C. L., Hill, J. A., and Olson, E. N. (2002). Class II histone deacetylases act as signal-responsive repressors of cardiac hypertrophy. Cell 110, 479-488.
Zhao, M., Chow, A., Powers, J., Fajardo, G., and Bernstein, D. (2004). Microarray analysis of gene expression after transverse aortic constriction in mice. Physiological genomics 19, 93-105.
Zhong, W., Mao, S., Tobis, S., Angelis, E., Jordan, M. C., Roos, K. P., Fishbein, M. C., de Alboran, I. M., and MacLellan, W. R. (2006). Hypertrophic growth in cardiac myocytes is mediated by Myc through a Cyclin D2-dependent pathway. EMBO J 25, 3869-3879.
Zuber, J., Shi, J., Wang, E., Rappaport, A. R., Herrmann, H., Sison, E. A., Magoon, D., Qi, J., Blatt, K., Wunderlich, M., et al. (2011). RNAi screen identifies Brd4 as a therapeutic target in acute myeloid leukaemia. Nature 478, 524-528.

Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of treating cardiomyopathy comprising administering to a subject in need to such treatment an amount of a BET inhibitor effective to treat the cardiomyopathy.

2. The method of claim 1, wherein:
(i) the subject does not have heart failure,
(ii) the subject is free of symptoms of obstructive coronary artery disease,
(iii) the subject is not being treated for atherosclerosis,
(iv) the subject does not have obstructive coronary artery disease, as evidenced by an angiogram showing,
(v) the subject does not have heart failure or atherosclerosis and is not recovering from a myocardial infarction, or
(vi) the subject is receiving therapy for reducing blood pressure.

3-7. (canceled)

8. The method claim 1, wherein the cardiomyopathy is due to chronic hypertension, valvular heart disease (aortic valve stenosis, aortic valve insufficiency, mitral valve insufficiency), peripartum cardiomyopathy, or cardiomyopathy due to genetic mutations, or wherein the cardiomyopathy is cardiac hypertrophy.

9. The method of claim 1, wherein the BET inhibitor is JQ1.

10. (canceled)

11. A method for treating heart failure not arising from inflammation comprising administering to a subject in need of such treatment an amount of a BET inhibitor effective to treat the heart failure.

12. The method of claim 11, wherein the subject does not have a obstructive coronary artery disease, as evidenced by an angiogram showing, or wherein the subject is not recovering from a myocardial infarction, or wherein the subject is not recovering from a myocardial infarction, or wherein the subject is receiving therapy for reducing blood pressure.

13-14. (canceled)

15. The method of claim 11, wherein the heart failure is due to:
(i) Heart failure with preserved ejection fraction (HFpEF) without evidence of obstructive coronary artery disease;
(ii) Heat failure due to toxicity of drugs (including anti-cancer agents and drugs of abuse);
(iii) Heart failure caused by ethanol abuse;
(iv) Heart failure due to chronic tachycardia (rapid heart rate);
(v) Heart failure due to endocrine abnormalities (excessive thyroid hormone, growth hormone, diabetes, pheochromocytoma);
(vi) High-output heart failure (includes that which is caused by anemia or peripheral atriovenous shunting);
(vii) Heart failure caused by nutritional deficiencies (including thiamine, selenium, calcium, and magnesium deficiency);
(viii) Heart failure due to viral infection (including HIV) or
(ix) Heart failure due to congenital heart malformations.

16. (canceled)

17. The method of claim 11, wherein the BET inhibitor is JQ1.

18. A method for treating myocardial infarction comprising administering to a subject in need of such treatment a BET inhibitor in an amount effective to treat the myocardial infarction, wherein the BET inhibitor administration is initiated not sooner than 5 days after the myocardial infarction.

19-20. (canceled)

21. The method of claim 18, wherein the subject does not have atherosclerosis as evidenced by an angiogram showing or wherein the subject does not have heart failure.

22. (canceled)

23. The method of claim 18, wherein the BET inhibitor is JQ1.

24. A method for cardio protection comprising administering to a subject receiving a therapy that is cardio toxic a BET inhibitor in an amount effective to inhibit cardio toxicity by such therapy.

25. The method of claim 24, wherein the therapy is anti-cancer therapy.

26. The method of claim 25, wherein the anti-cancer therapy is chemotherapeutic therapy.

27. (canceled)

28. The method of claim 24, wherein the BET inhibitor is JQ1.

29. A method for inhibiting restenosis comprising administering to a subject undergoing an angioplasty and/or receiving a stent a BET inhibitor in an amount effective to inhibit restenosis.

30. The method of claim 29, wherein the BET inhibitor is administered locally at the site of a stenosis.

31. The method of claim 30, wherein the BET inhibitor is administered via a catheter or as an element of a coating on a stent.

32. (canceled)

33. The method of claim 29, wherein the BET inhibitor is JQ1.

34. In a stent for preventing stenosis or restenosis, the stent including a coating for delivering a drug agent locally to the vasculature when the stent is positioned at the vasculature, the improvement comprising a BET inhibitor included in the coating.

35. The stent of claim 34, wherein the BET inhibitor is JQ1.
